(19) United States
(12) Patent Application Publication
(10) Pub. No.: US 2018/0129072 A1
(43) Pub. Date: May 10, 2018

(54) CONTACT AND INTRAOCULAR LENSES COMPRISING AN ADJUSTABLE FOCUS LENGTH

(71) Applicant: Optotune AG, Dietikon (CH)

(72) Inventors: Manuel ASCHWANDEN, Allenwinden (CH); David NIEDERER, Kuttigen (CH); Stephan SMOLKA, Zurich (CH); Chauncey GRATZEL, Palo Alto, CA (US); David STADLER, Zurich (CH); Roman PATSCHEIDER, Winterthur (CH); Matthias WALSER, Zurich (CH); Alexandre LARMAGNAC, Widen (CH); Marcel SUTER, Zurich (CH); Thomas SCHMIDHAUSLER, Tuggen (CH)

(73) Assignee: Optotune AG, Dietikon (CH)

(21) Appl. No.: 15/570,329

(22) PCT Filed: Apr. 28, 2016
(86) PCT No.: PCT/EP2016/059572

§ 371 Date: Oct. 29, 2017

(30) Foreign Application Priority Data
Apr. 28, 2015 (EP) PCT/EP2015/059113

(51) Int. Cl. G02C 7/04; B29D 11/00; A61F 2/16; G02B 3/14; G02C 7/08

(52) U.S. Cl. CPC G02C 7/049; B29D 11/00048; G02C 7/085; B29D 11/00817; G02B 3/14; A61F 2/1635

(57) ABSTRACT

The invention relates to a lens (1) for vision correction, wherein the lens (1) is configured to be placed directly on the surface of an eye (2) of a person or to be implanted into an eye (2) of a person, and wherein the lens (1) further comprises: a transparent base element (10) having a back side (12) and a front side (11) facing away from the back side (12), a transparent and elastically expandable membrane (20) connected to said base element (10), wherein said membrane (20) comprises a back side (22) that faces said front side (11) of the base element (10), a ring member (30) connected to said back side (22) of the membrane (20) so that the ring member (30) defines a curvature-adjustable area (23) of the membrane (20), and wherein the lens (1) comprises a lens volume (41) adjacent said curvature-adjustable area (23) of the membrane (20), which lens volume (41) is delimited by the ring member (30), and wherein the lens (1) comprises a reservoir volume (42) adjacent a boundary area (24) of said membrane (20), wherein said two volumes (41, 42) are filled with a transparent liquid (50), and wherein said volumes (41, 42) are fluidly connected or fluidly connectable to each other such that, when the reservoir volume (42) is compressed, liquid (50) residing in the reservoir volume (42) is pressed into the lens volume (41) such that the curvature of said curvature-adjustable area (23) of the membrane (22) increases and the focal length of the lens (1) decreases. Further, the invention relates to a method for manufacturing a contact lens according to the invention.

The present invention relates to a lens, particularly a contact lens or an intraocular lens, having an adjustable focal length.

More particularly, the present invention relates to designs and methods of how to use and control such dynamic lenses. The present invention is not only applicable to contact lenses or intraocular lenses that are to be implanted into an eye but also to other lenses that may be used in a variety of different applications.

One particular aspect of the present invention shows how excellent optical quality can be achieved using liquid filled membrane lenses while employing actuation systems or a control system that consume little or no power, particularly no external power. Furthermore, an aspect of the present invention relates to a method for charging an energy source for the lens, particularly for a control system of the lens. Yet another aspect of the present invention relates to different methods for controlling the focal power or focal length of the lens. Further, a method to detect an input signal from the user is described. Particularly, some aspects of this invention aim at implementing a deformable contact or intraocular lens which allows correction of refractive and/or accommodation deficiencies of the eye of the user to deliver particularly high optical qualities. Furthermore, an aspect of the present invention relates to the control of focal power of the lens by means of a movement of the respective eyelid, wherein particularly a fast blinking motion of the eye lid can be decoupled from the focal power control movement of the eyelid, particularly be means of an (e.g. mechanical) low pass filter. Further, a method to control the time constants of the said low pass filter is described.

In WO2008115251 a soft contact lens is described that has a body with a central zone aligned with the optical axis of the eye when a user wears the lens. In one embodiment the soft lens includes a chamber that extends from a lower portion of the lens to its central axis and is arranged such that when a person looks down, a fluid is squeezed from the reservoir and changes the optical characteristics of the lens.

Further, WO98/14820 describes a variable focus contact lens, which has a body with a first half and an opposite second half. The body also has a first peripheral surface, an opposite second peripheral surface and an associated focal length. The lens includes a first material that is resilient so that when a compressive force is applied to the first surface and the second surface, the focal length of the lens changes in proportion to the compressive force. A force-distributing structure is disposed for distributing forces within the lens so as to inhibit astigmatism in the lens.

Furthermore, the fluid-filled adjustable contact lens of US 2012/0268712 shows an exemplary contact lens which includes a lens chamber configured to be positioned on a pupil of a user wearing the contact lens; a reservoir fluidly connected to the lens chamber, an actuator configured to transfer fluid back and forth between the lens chamber and the reservoir; a sensor configured to sense movement from the user and transmit a control signal when a predetermined movement is performed by the user, and a processor configured to actuate the actuator upon receipt of the control signal from the sensor.

Further, U.S. Pat. No. 8,755,124 describes an adjustable optical lens comprising a membrane, a support for the membrane, a fluid between the membrane and the support, an actuator for deforming the membrane, and a rigid ring connected to the membrane surrounded by the rigid ring where the rigid ring has a defined circumference.

Based on the above, the problem underlying the present invention is to provide an improved contact lens that particularly allows to precisely adjust the focal length of the contact lens and achieves a high optical quality.

This problem is solved by a contact lens having the features of claim 1. Preferred embodiments of the present invention are stated in the corresponding sub claims or are described below.

According to claim 1, the adjustable focus length lens is configured to be placed directly on the surface of an eye of a person (e.g. covering the pupil of said eye) or to be implanted into an eye of a person, and wherein the lens further comprises:
a transparent base element having a back side and a front side facing away from the back side,
a transparent and elastically expandable membrane connected to said base element, wherein said membrane comprises a back side that faces said front side of the base element,
particularly a ring member (or a ring structure) connected to said back side of the membrane so that the ring member (or structure) defines a curvature-adjustable area of the membrane, and
wherein the lens comprises a lens volume adjacent said curvature-adjustable area of the membrane, which lens volume is delimited by the ring member, and wherein the lens comprises a reservoir volume adjacent a boundary area of said membrane, wherein said two volumes are filled with a transparent liquid, and
wherein said volumes are fluidly connected or fluidly connectable to each other such that, when the reservoir volume is compressed, liquid residing in the reservoir volume is pressed into the lens volume such that the curvature of said curvature-adjustable area of the membrane increases and the focal length of the lens decreases.

According to an embodiment, the lens is a contact lens. In this case, the base element may be configured to be placed directly on the surface of the eye of a person such that the back side of the base element contacts the eye. In an alternative embodiment it is also possible that the membrane is configured to contact the eye (with the front side of the membrane facing away from the back side of the membrane). Here, the incident light first passes through the base element then passes through the lens volume and finally through the membrane (i.e. through the curvature-adjustable area) before entering the eye on which it is placed.

Generally the transparent liquid can also be a transparent fluid. In some embodiments a fluid resides in the reservoir(s) and/or reservoir volume and/or lens volume and is used to adjust the curvature of the curvature-adjustable area. However, such a fluid can also be a liquid, particularly a transparent liquid.

Particularly, said ring member separates said lens volume adjacent or below the curvature-adjustable area of the membrane from the reservoir volume adjacent or below said boundary area of the membrane.

Further, particularly, the ring member may be integrally formed with the membrane and may protrude from said back side of the membrane.

Particularly, said curvature adjustable-area of the membrane is configured for passing light through the curvature adjustable-area which deflects the light passing through it according to the current curvature of said area of the membrane. Particularly, said curvature-adjustable area corresponds to the clear aperture of the lens according to the invention.

Further, particularly, the base element may form a base lens. Furthermore, particularly, the base element is stiffer than the membrane. Likewise, the ring member is preferably stiffer than the membrane so as to be able to define the shape of the lens (i.e. of said curvature adjustable area). Particularly, said ring member is a circular ring member.

Furthermore, according to an embodiment of the lens according to the invention, the back side of the base element comprises a concave curvature so that the back side of the base element can fully contact the eye of a person.

Particularly, the base element can consist of or comprise one of the following materials:
A glass,
Polymers including elastomers (e.g. TPE, LCE, Silicones, e.g. PDMS, acrylics, urethanes),
A Plastic including thermoplasts (e.g. ABS, PA, PC, PMMA, PET, PE, PP, PS, PVC) and duroplasts,
A Gel (e.g. silicone hydrogel, polymacon or optical gel OG-1001 by Liteway).

Particularly, the membrane can consist of or comprise one of the following materials:
A glass,
A polymers including elastomers (e.g. TPE, LCE, Silicones, e.g. PDMS, acrylics, urethanes),
A plastic including thermoplasts (e.g. ABS, PA, PC, PMMA, PET, PE, PP, PS, PVC) and duroplasts.
A gel (e.g. silicone hydrogel, polymacon or optical gel OG-1001 by Liteway),

Further, particularly, the liquid can be or comprise one of the following substances: a fluorinated silicone, water, an ionic liquid, ionic gels, a silicone, a contact lens cleaning solution, a salty water solution, an oil, a solvent.

According to an embodiment of the present invention, the lens volume is configured to be compressed, wherein when the lens volume is compressed, liquid residing in the lens volume is pressed into the reservoir volume such that the curvature of said curvature-adjustable area of the membrane decreases and the focal length of the lens increases.

According to an embodiment of the present invention, the reservoir volume is fluidly connected or fluidly connectable with the lens volume via at least one opening. Fluidly connected means that there exists a flow connection such that liquid can pass via said connection from the lens volume to the reservoir volume and vice versa.

Further, according to an embodiment of the present invention, the at least one opening is a circumferential gap defined by a face side of the ring member (which face side faces the front side of the base element) and the base element, wherein particularly, when the curvature-adjustable area of the membrane assumes a maximal convex curvature, said face side of the ring member contacts the front side of the base element.

Further, according to an embodiment of the present invention, the ring member is also connected to the front side of the transparent base element, particularly via its face side.

Particularly, the at least one opening is a channel extending (e.g. in or along a radial direction) through the ring member so that a flow connection, particularly a permanent flow connection, is established between the lens volume and the reservoir volume. According to a further embodiment, the ring member may also comprise a plurality of openings in the form of channels that fluidly connect the reservoir volume to the lens volume and that particularly extend in or along a radial direction through the ring member.

Further, said openings or channels may be delimited by the ring member and by the front side of the base element to which the ring member is attached, particularly via its face side facing the front side of the base element. Here, the openings can be formed by forming recesses into the edge or face side of the ring member to that channels result when the ring member is connected with its face side to the front side of the base element.

In the above embodiments, one or more dimensions of one opening or channel or said plurality of openings and channels, are (e.g. mechanically or electrically) controllable.

In other words, one opening or channel or said plurality of openings and channels can act as static and/or dynamic flow and/or pressure regulators (e.g. check-valves, regulating valves, or regulating flow resistors).

Further, particularly, one or more dimensions of one opening or channel or said plurality of openings and channels, are modulated prior, during, and/or after each or only selected eye blinks.

In other words, the fluid exchange between the reservoir volume and the lens volume is modulated synchronously with the eye blinking to enable, enhance, and/or suppress curvature changes of the lens (e.g. at least over a pre-defined time-period). For example, the flow and/or pressure resistance during an actuation movement is reduced, and/or the flow and/or pressure resistance between subsequent actuation movements is increased. In the above embodiments, the dimensions of the at least one opening or said plurality of openings are chosen particularly such that a time period over which the reservoir volume or the lens volume have to be compressed in order to yield a change of the curvature of the curvature-adjustable area is longer than the blink of an eye lasts, particularly longer than 1 second, particularly longer than 0.9 seconds, particularly longer than 0.8 seconds, particularly longer than 0.6 seconds, preferably longer than 0.5 seconds.

In other words, in case the openings or channels between the lens volume (e.g. optically clear aperture) and the reservoir volume are sufficiently small, an eye blinking movement of the person wearing the (e.g. contact) lens will be low-pass filtered and will thus not change the curvature of the lens. Only a slow enough actuation movement will result in a change of the focal power of the (e.g. contact) lens. Further, according to an embodiment of the present invention, the reservoir volume is configured to be compressed by an eyelid of an eye of the person when the (e.g contact) lens is arranged on the pupil of said eye, wherein particularly the reservoir volume is arranged such in the lens that the reservoir volume is compressed and the curvature of the central area of the membrane increases, when said person closes said eyelid partially [e.g. at least over a pre-defined time period).

The lens is particularly configured to maintain a compressed state of the reservoir. Such a state can be released e.g. by pushing on the lens volume.

Therefore, according to an embodiment of the present invention, the lens volume is configured to be deformed or compressed by the eyelid of the person when the contact lens is arranged on the pupil of the corresponding eye, particularly by closing said eyelid so as to press liquid from the lens volume back into the reservoir volume.

By selecting the geometries of the reservoir and lens volume appropriately, the overall change of the lens volume during the blinking of the eye is substantially zero. Here, substantially zero means that the focal power of the lens changes by not more than 0.25 diopter, and in particular not more than 0.1 diopter, and in particular not more than 0.05 diopter.

According to an embodiment, the reservoir volume is delimited by a first surface formed e.g. by the membrane and by a second surface formed e.g. by the base element, wherein said surfaces face each other, and wherein particularly said surfaces are configured to stick to each other (e.g. passively, e.g. due to adhesion forces, or actively, e.g. electrostatically) when making contact upon compression of the reservoir volume such that a compressed state of the reservoir volume can be maintained.

Further, the said stiction can be used to seal the opening and/or channels connecting the reservoir volume and lens volume.

Further, according to an embodiment of the present invention, the lens comprises at least one actuator that is configured to compress the reservoir volume so as to press liquid from the reservoir volume into the lens volume.

Further, particularly, according to an embodiment of the present invention, the curvature-adjustable area of the membrane is configured to act as a spring (and mechanical energy source) so that liquid can be pushed back from the lens volume into the reservoir volume, e.g. when at least one or said plurality of actuators and/or regulators stop acting, particularly compressing, the reservoir volume and/or stop acting on the openings or channels connecting the reservoir volume and the lens volume (e.g. when the reservoir volume is released).

Further, according to an embodiment the present invention, the lens is configured to regulate and/or completely hinder said pushing back of liquid from the lens volume into the reservoir volume by closing and/or sealing at least one or said multiple of openings or channels.

Further, according to an embodiment of the present invention, the reservoir volume is delimited by a first surface formed e.g. by the membrane and a second surface formed e.g. by the base element, wherein the two surfaces face each other.

Further, according to an embodiment of the present invention, the actuator comprises a particularly compliant (first) electrode (i.e. a flexible conducting element) attached to said first surface and an insulated (second) electrode (rigid or flexible conducting element) attached to said second surface such that an e.g. tapered gap is formed between the electrodes, wherein, when a voltage is applied to said electrodes said gap is reduced by an amount depending on the magnitude of the applied voltage and liquid is pressed from the reservoir volume (e.g. out of said gap) into the lens volume. Of course also the first electrode or both electrodes can be insulated. It is merely advantageous to insulate the electrodes with respect to each other.

Further, according to an embodiment of the present invention, the electrodes of the actuator are split up into individual sections forming pairs of electrodes that are configured to be actuated individually in a discrete or in a continuous manner. Discrete means that two electrodes forming a pair are either apart from each other forming a gap or contact each other (no gap). Thus a discrete amount of liquid can be transferred between said volumes by such a pair of electrodes depending on the size of the gap. Continuous means that the gap between two electrodes is closed continuously so that an adjustable amount of liquid can be transferred between said volumes. Particularly, an actuator that comprises the afore-described electrodes, pair of electrodes or corresponding segments or sections is also denoted as zipper or zipping actuator herein.

Further, according to an embodiment of the present invention, the lens is configured to use certain individual sections of said electrodes to (particularly passively) control fluid pressures and fluid flow rates for controlling the time periods upon which the fluid exchange between the reservoir volume and the lens volume takes place. In particular, the lens is configured to increase the flow and/or pressure resistance, and/or to completely suppress the fluid flow by closing and/or sealing at least one or multiple of said electrode sections.

Further, particularly, the center of the lens (i.e. curvature-adjustable area) is configured to act as a spring that wants to open (e.g. unzip) the actuator(s), i.e. move the first and second electrode(s) apart from each other corresponding to the open state of the actuator in contrast to a closed state where the respective first and second electrode contact each other and the associated gap vanishes in particular. Of course, the gap can also take any size between the said open and the said closed state. Further, the gap can be spatially varying, e.g. the first and second electrode can contact each other only at a certain percentage of the whole electrode area, whereas other areas remain in the open state. Such a partially closed/zipped state can be addressed by controlling the actuator force, in particularly, by controlling the actuator voltage.

According to an embodiment of the present invention, at least one or several non-linear elements (e.g. check-valves, friction elements, resonant cavities) are integrated into e.g. the fluid reservoir(s) or reservoir volume, channels, or actuator regions, to address various well defined actuator states (e.g. closed, partially closed by a certain percentage, or open). In contrast to above said actuator voltage control, non-linear elements can be used to address various actuator states without the need to control the actuator force. For example, the actuator state can be controlled by volume, e.g. by fully depleting a reservoir of fixed volume, or by pressure, e.g. by using a check-valve that opens at a specific pressure level)

Further, according to an embodiment of the present invention, the electrodes or the insulation layers can be modified (e.g. coated, micro-structured, chemically modified) such that they stick less, or do not stick, or do stick with a specific stiction force to each other when making contact. In other words, the threshold voltages for actuation can be reduced or stabilized by said surface modifications.

Further, according to an embodiment of the present invention, the said sticktion can be temporarily or permanently lowered or regulated by e.g. pressure waves (e.g. blinking caused pressure fluctuations, ultrasonic transducers), and/or alternating electrostatic forces (AC signals applied to the said or additional electrodes).

In other words, rapid eye-lid movements and/or AC voltage modulations (e.g. at the system resonant frequency) can assist the separation and/or approaching of the said actuator and/or regulator first and second electrodes, thus effectively lowering the voltage and/or energy required to access individual equilibrium states.

Further, according to an embodiment of the present invention, individual equilibrium states are connected such that energy (e.g. mechanical or electrical) from one state can be temporarily stored and transferred to another state (e.g. forming at least a bi-stable system or a system with multiple equilibrium states)

Further, according to an embodiment of the present invention, for reducing an influence of an eyelid on the reservoir volume and said electrodes, the reservoir volume is arranged next to the lens volume in a horizontal direction when the lens is arranged with respect to an eye as intended (in relation to an upright position of the head of the user).

Further, according to an embodiment of the present invention, the at least one actuator extends circumferentially around the ring member.

Further, according to an embodiment of the present invention, the ring member is at least 5 times, particularly at least 10 times, particularly at least 50 times, particularly at least 100 times, particularly at least 1000 times stiffer than the membrane.

Further, according to an embodiment of the present invention, the ring member has a circularity and flatness better than 25 μm, particularly better than 10 μm, particularly better than 5 μm at an interface between the ring 20 member and the membrane.

Further, according to an embodiment of the present invention, the lens comprises a sensor configured to sense a movement from the person wearing the lens, and to provide an output signal in response to a pre-determined movement of the user, wherein particularly said movement is a movement of an eyelid of an eye of said person, on which eye said contact lens is arranged

Further, the lens particularly comprises a processing unit that is configured to actuate the at least one actuator in response to the output signal provided by the sensor or in response to an output signal provided by an external device, wherein particularly the at least one actuator is actuated by applying said voltage or voltages to said electrodes of the at least one actuator as described above (e.g. for opening and closing gaps between associated first and second electrodes).

According to an aspect of the present invention a system may be provided comprising a lens according to the invention and an external device configured to provide said output signal.

Further, according to an embodiment of the present invention, said sensor is one of: a photosensitive element, a pressure sensing element, a capacitive sensing element, a thermal sensor, particularly a resistor. Particularly said resistor may extend along the periphery of the contact lens. When the person covers the resistor with an eyelid, the temperature of the resistor rises due to heat transferred from the eyelid to the resistor.

Further, according to an embodiment of the present invention, the contact lens comprises an electric energy source, particularly a battery.

Further, according to an embodiment of the present invention, said electric energy source is configured to be charged by means of one of:
inductive charging;
current generation by means of an reverse electro-osmotic effect;
light, wherein particularly the contact lens comprises a solar cell or a photo diode;
using the thermoelectrical effect, wherein particularly the contact lens comprises a Peltier element;
electrostatic charging (e.g. charging of surface layers);
piezo-electric resonators (e.g. charged by human voice) harvesting eye lid movements, wherein particularly the contact lens comprises a flexible capacitance for transforming eye lid movements into electrical energy that can be stored in said energy source/battery.

Further, according to an embodiment of the present invention, said surfaces (e.g. of the membrane and base element, see above) are configured to stick to each other through a compressive force of the at least one actuator, meaning for instance that they are configured to stick to each other when brought to contact each other by means of the at least one actuator.

Further, according to an embodiment of the present invention, the back side of the base element is configured to be placed on the surface of the eye such that said back side contacts said surface of the eye, or that the front side of the membrane is configured to be placed on the surface of the eye such that said front side contacts said surface of the eye. Also in case of an intraocular lens, either the base element or the membrane may be configured to be passed first by incident light that hits the eye.

Further, according to an embodiment of the lens according to the invention, the reservoir volume is positioned in an upper half of the lens (or alternatively in a lower half for the lower eyelid), so that the reservoir volume is compressible by an onset of an upper (or lower) eyelid movement of an eye of the person when the lens is arranged on the pupil of said eye, so as to pump liquid from the reservoir volume into the lens volume for increasing the curvature of the curvature-adjustable area of the membrane.

Further, according to an embodiment of the lens according to the invention, the reservoir volume is formed by at least one, particularly two, or even more separate reservoirs which are each arranged in said upper (or lower) half and can each be brought in flow connection to the lens volume via a respective channel extending along a periphery of the lens volume from the upper half of the lens to the lower half of the lens.

Further, according to an embodiment of the lens according to the invention, said at least one or several channels are connectable to the lens volume via one or several valves which valve(s) is/are arranged in a lower (or upper) half of the lens so that the respective valve faces the reservoirs and/or so that the lens volume is arranged between the reservoirs and the valve(s).

Further, according to an alternative embodiment of the lens according to the invention, each reservoir comprises a valve via which the respective reservoir is connected to its associated channel, wherein the respective valve may comprises an osmotic membrane forming a wall (particularly bottom) of the respective reservoir, which osmotic membrane opens and allows the liquid to pass through it when a suitable voltage is applied to the osmotic membrane.

Alternatively, the respective valve may be one of the following valves: a valve comprising at least two electrodes for opening or closing the valve (e.g. a zipper or zipping actuator as described herein); a valve comprising a member out of a shape memory alloy or a phase change material for opening or closing the valve; a valve comprising an electromagnetic actuator for opening or closing the valve; a valve comprising a magnet that is configured to be moved by a another magnet for opening or closing the valve (e.g. an external magnet).

Further, according to an embodiment of the lens according to the invention, the lens comprises an energy source that is electrically connected to the valve via a power line for providing energy to the valve in order to open or close the valve.

Further, according to an embodiment of the lens according to the invention, the lens comprises a sensor for detecting an eyelid movement, which sensor is connected to the valve or the energy source via a data line, wherein the sensor is configured to provide an output signal when an eyelid movement is detected by the sensor and to provide the output signal to the valve or energy source via said data line for controlling the valve, particularly for closing or opening said valve.

Further, according to yet another embodiment of the lens according to the invention, the lens comprises a pump which comprises the reservoir volume, wherein the pump is configured to empty the reservoir volume by moving (e,g. pulling or pushing) a region of said membrane covering the reservoir volume into a dent of the base element forming at least a part of said reservoir volume.

Further, according to an embodiment of the lens according to the invention, said dent may comprise a concave shape (or a conical shape or some other suitable geometry)

Further, according to an embodiment of the lens according to the invention, the reservoir geometry is designed such that minimal or no energy is used to move (e.g. pull or push) said region of the membrane into the dent of the reservoir volume.

Further, according to an embodiment of the lens according to the invention, the pump is configured to generate an electrostatic force for pulling said region of the membrane into the dent of the reservoir volume, wherein for generating said force said region of the membrane comprises a flexible and particularly stretchable, electrically conducting electrode, and the base element comprises at least one corresponding counter electrode facing said electrode of the membrane.

Alternatively the valve may comprise a member out of a shape memory alloy that may be configured to expand upon heating (e.g. by means of an electrical current) and then moves said region of the membrane into the dent. Further, according to an embodiment of the lens according to the invention, dielectric layers may be applied either on both, the region of the membrane and the base element, or only on the base element.

Further, according to an embodiment of the lens according to the invention, a channel (e.g. in the form of a groove formed in the base element) via which the reservoir volume is connected to the lens volume leads to a particularly lowest (e.g. central) area of a bottom of said dent of the reservoir volume for draining said dent, wherein said groove is configured to be automatically sealed when said region of the membrane is moved (e.g. pulled or pushed) into the dent.

Further, according to an embodiment of the lens according to the invention, The amount of liquid transferred is properly defined by the reservoir volume. Several reservoir volumes can be combined to transfer fluid in discrete steps.

Further, according to an embodiment of the lens according to the invention, when said channel (or groove) is sealed, re-entry of liquid into the reservoir volume is blocked at an intersection of the channel/groove and the reservoir volume which intersection is also denoted as sealing line.

Further, according to an embodiment of the lens according to the invention, the pump is configured to keep the channel that forms a valve here in its sealed or closed state by pinning said region of the membrane to an (e.g. central) area on the bottom of said dent of the reservoir volume (this area is also denoted as sealing area and can be identical to said lowest area of the dent) using the electrode of the membrane on one side and on the other side said counter electrode and/or a central electrode that is arranged at the center of the bottom of the dent and surrounded by said counter electrode. Alternatively the member (shape memory alloy) may be used to pin down the membrane region.

Further, according to an embodiment of the lens according to the invention, the active electrode area and the electric power can be reduced after pinning the membrane to the bottom of the dent/reservoir volume. Further, the (e.g. circular) sealing area can be flexible, stiff, or even rigid.

Further, according to an embodiment of the lens according to the invention, particularly depending on the electric power applied, the sealed channel is configured to open at a certain back pressure, which initiates liquid back flow and refilling of the reservoir volume.

Further, according to yet another embodiment of the lens according to the invention, the lens comprises a channel for providing a flow connection between the reservoir volume and the lens volume, wherein the lens comprises a valve for opening or closing said channel, wherein said channel extends through a dent of the valve formed in the base element, which dent is covered by a region of said membrane, wherein the valve is configured to open or block said channel by moving (e.g. pulling or pushing) a region of said membrane covering the dent into the dent.

Further, according to an embodiment of the lens according to the invention, the dent geometry is designed such that minimal or no energy is used to move (e.g pull or push) the membrane into the dent.

Further, according to an embodiment of the lens according to the invention, the valve is configured to generate an electrostatic force for pulling said region of the membrane into the dent of the valve, wherein for generating said force said region of the membrane comprises a flexible and particularly stretchable, electrically conducting electrode, and the base element comprises at least one corresponding counter electrode. Alternatively, the valve may comprise a member out of a shape memory alloy for generating said force (see also above).

Further, according to an embodiment of the lens according to the invention, dielectric layers may be in turn applied either on both, the region of the membrane and the base element, or only on the base element.

Further, according to an embodiment of the lens according to the invention, said channel is configured to be automatically blocked when said region of the membrane is moved (e.g. pulled or pushed) into the dent of the valve.

Further, according to an embodiment of the lens according to the invention, when said channel is blocked, re-entry of liquid into the dent of the valve is blocked at intersections of the channel and the dent, which intersections are also denoted as sealing lines. Particularly, there are two such intersections or sealing lines, one where the channel enters the dent, and a further one where it leaves the dent.

Further, according to an embodiment of the lens according to the invention, the valve is configured to keep the channel in its blocked state by pinning said region of the membrane to an (e.g. central) area on the bottom of said dent of the valve (this area is also denoted as sealing area and can be identical to a lowest area of the dent) using the electrode of the membrane on one side and on the other side said counter electrode and/or a central electrode that is arranged at the center of the bottom of the dent and surrounded by said counter electrode and/or a first and/or a second sealing line electrode which extend along the sealing lines and are separated from the central electrode by a gap.

Further, according to an embodiment of the lens according to the invention, the active electrode area and the electric power can be reduced after pinning the membrane to the bottom of the dent/reservoir volume. Further, the (e.g. circular) sealing area can be flexible, stiff, or even rigid.

Further, according to an embodiment of the lens according to the invention, depending on the electric power applied, the valve is configured to open at a certain pressure, which allows passage of liquid between the reservoir volume and the lens volume via the channel.

In the embodiments described above where liquid is moved by means of an actuator (e.g. a pump), the membrane or at least a region thereof is configured to be pushed down by an eyelid of a user of the lens in order to assist in pumping liquid from the reservoir volume to the lens volume and/or from the lens volume into the reservoir volume, or in order to assist to close at least one or several valves of the lens.

Further, according to yet another embodiment of the lens according to the invention, the reservoir volume is covered by a bistable region of said membrane, wherein said region is movable with respect to the base element from a first stable state to a second stable state and vice versa, wherein in the first state, the reservoir volume is larger than in the second state, and wherein when said region is moved from the first state to the second state, liquid flows from the reservoir volume into the lens volume, and wherein when the region is moved from the second state to the first state, liquid flows from the lens volume back to the reservoir volume.

Further, according to an embodiment of the lens according to the invention, the lens comprises a channel connecting the reservoir volume to the lens volume to allow liquid to flow from the lens volume to the reservoir volume and vice versa.

Further, according to an embodiment of the lens according to the invention, the reservoir volume comprises a circular shape or a ring shape extending around the lens volume.

Further, according to an embodiment of the lens according to the invention, said bistable region of the membrane is configured to flip from one stable state to the other stable state when sufficient pressure is applied to a concave or convex surface of said region, wherein said region is configured to be actuated manually (e.g. by a person) in order to move it from one state to the other, particularly by means of a finger or an eyelid of a person.

Further, according to an embodiment of the lens according to the invention, said bistable region of the membrane is given a convex or concave shape using molding or thermoforming for providing said bi-stable state.

Further, according to an embodiment of the lens according to the invention, said region of the membrane is made out of an elastomer.

Further, according to an embodiment of the lens according to the invention, a portion of the membrane or said region of the membrane is made out of metal, or polymer, or an elastomer, or a heterogeneous structure of at least two materials. For example: a disk of Kapton embedded in silicone.

According to a further aspect of the present invention, a system is disclosed comprising a lens according to the invention as described or claimed herein and a container for storing the lens when the lens is not placed on the surface of an eye of the user, wherein said container comprises an electrically conducting coil for charging a battery of the lens by means of induction, when the lens is arranged in the container. Here, particularly, the lens may comprise an electrically conducting coil, too, that is connected to the energy source (e.g. battery) of the lens.

According to a further aspect of the present invention, a method for manufacturing a contact lens, particularly according to the invention, having the features of claim 58 is proposed, comprising the steps of:
providing a base element (e.g. by way of molding, e.g. out of a silicone hydrogel, or a silicone coated with silicone hydrogel),
providing an elastically deformable membrane (e.g. by way of molding, e.g. out of a silicone hydrogel or a silicone coated with silicone hydrogel) comprising a ring member connected to a back side of the membrane,
bonding of the base element to the (e.g. back side of the) membrane and thereby forming a lens volume and a reservoir volume of the contact lens, and
filling said lens volume and said reservoir volume with a transparent liquid

Particularly, one of the following is applied to the membrane and/or the base element: a coating, at least one electrode, an insulation layer, an anti-stiction layer.

Particularly, the ring member can be plasma bonded to the membrane. Furthermore, the base element can be plasma bonded or glued to the membrane.

Further, particularly, the ring member can be integrally formed with the membrane (e.g. upon molding of the membrane), wherein the ring member can be stiffened by means of irradiating it with ultraviolet light or wherein the membrane can be softened by irradiating it with ultraviolet light. Materials that may be used for the ring member and membrane that can be stiffened by irradiating them with ultraviolet light are for example: silicones or urethanes. Further, materials that may be used for the membrane and ring member that can be softened by irradiating them with ultraviolet light are for example: silicones or urethanes).

Alternatively, a primer may be applied to the mold which is designed to chemically stiffen the ring member during molding of the membrane and integral ring member.

Further, according to an embodiment of the present invention, said filling is conducted using osmosis after said bonding has been performed.

For this, particularly, a pre-defined amount of water soluble salt is arranged on the base element or membrane before bonding so that said salt is arranged in the lens volume and/or lens reservoir after bonding, wherein then the bonded base element and membrane is soaked in the transparent liquid which enters the lens volume and reservoir volume by way of osmosis.

Further, according to an alternative embodiment of the present invention, said filling is conducted before said bonding, wherein said liquid is filled into a dent formed by the membrane, wherein thereafter said bonding is conducted, and wherein the lens volume and/or reservoir volume is freed from gas residing therein after said bonding.

Here, a glue, particularly a glue ring between the edge of the membrane and the edge of the base element, may be used, which glue is cured after freeing the lens volume/reservoir volume from said gas. This allows to adjust the initial focal length of the contact lens. Here, a glue that can be hardened by irradiating it with ultraviolet light may be used, wherein curing of the glue is then conducted by irradiating the glue with ultraviolet light after said degassing (i.e. freeing said volumes from the gas therein).

Furthermore, in an embodiment where filling is performed before bonding, the membrane may be provided (instead of molding) by vapor coating the liquid arranged on the base element by means of vapor depositing (coating) A material that can be used to vapor-deposit the membrane (the ring member is provided before (e.g. arranged on the base element) is e.g. parylene (i.e. chemically vapor deposited poly(p-xylylene) polymers).

The present invention can be used in a large variety of applications, such as contact lenses or intraocular lenses, or in any other lens that requires an adjustable focal length.

The invention will be better understood and objects other than those set forth above will become apparent when consideration is given to the following detailed description thereof. Such description makes reference to the drawings, wherein:

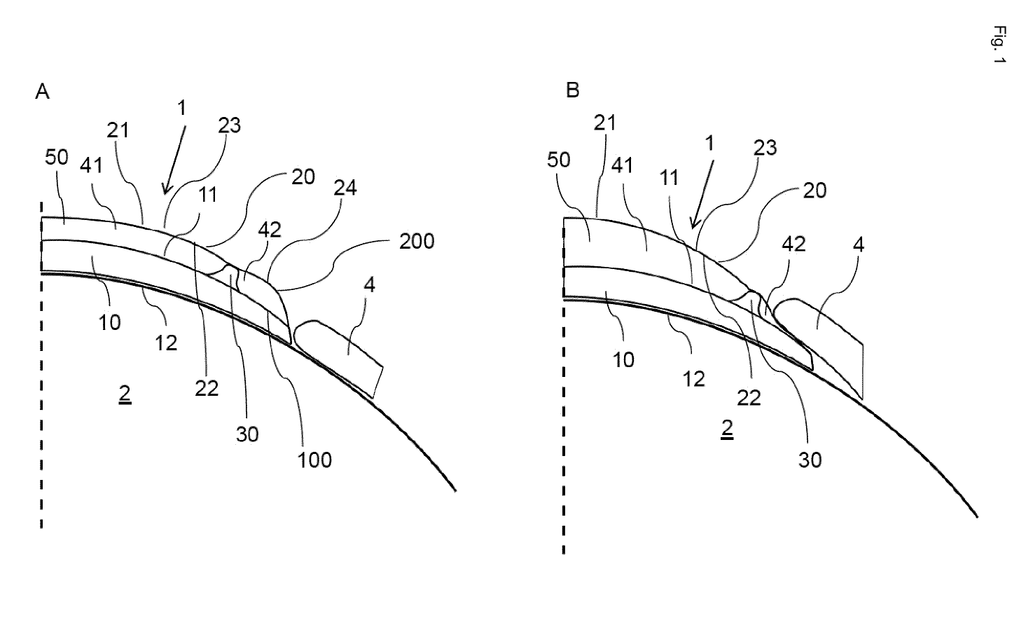

FIG. 1 shows an embodiment of a contact lens according to the present invention;

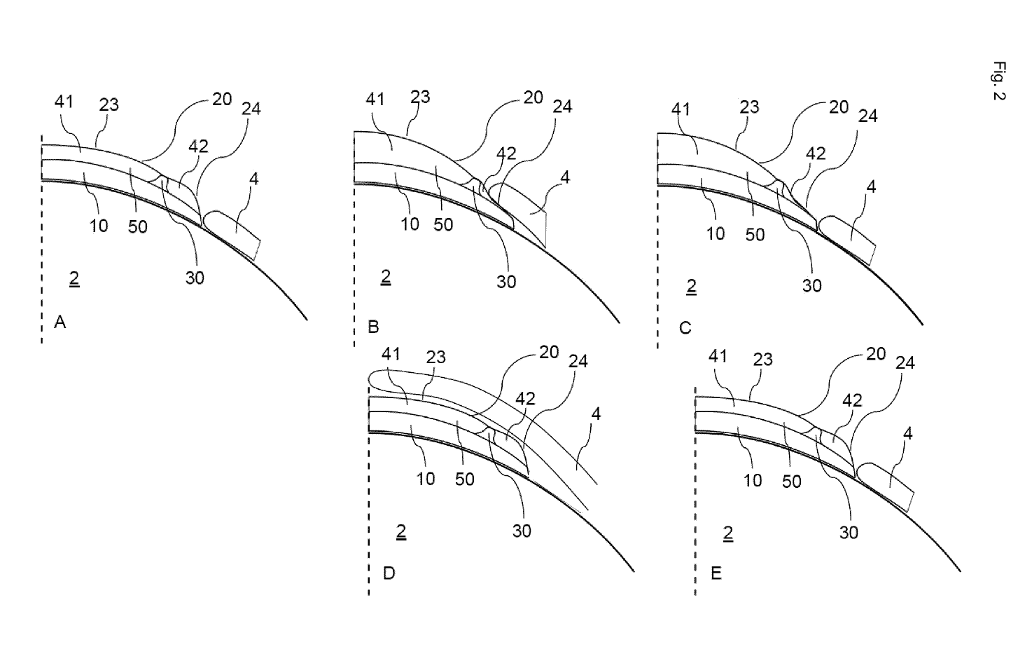

FIG. 2 shows an actuation of the contact lens according to FIG. 1 by means of an eyelid;

FIG. 3 shows two different variants of openings in the ring member for fluidly connecting the lens volume and the reservoir volume;

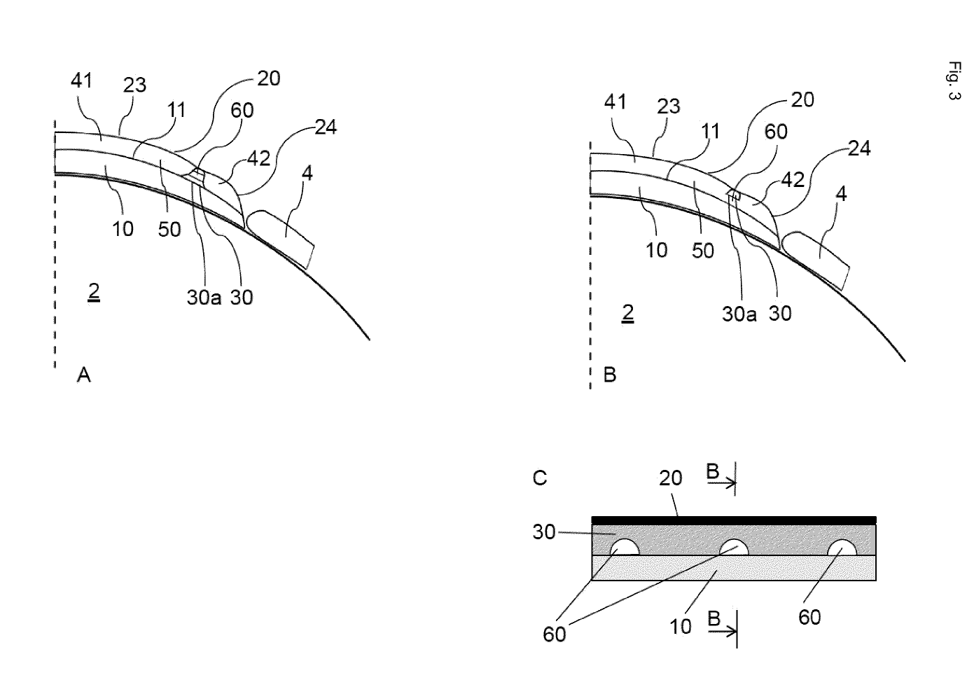

FIG. 4 shows an embodiment of a contact lens according to the present invention using an actuator;

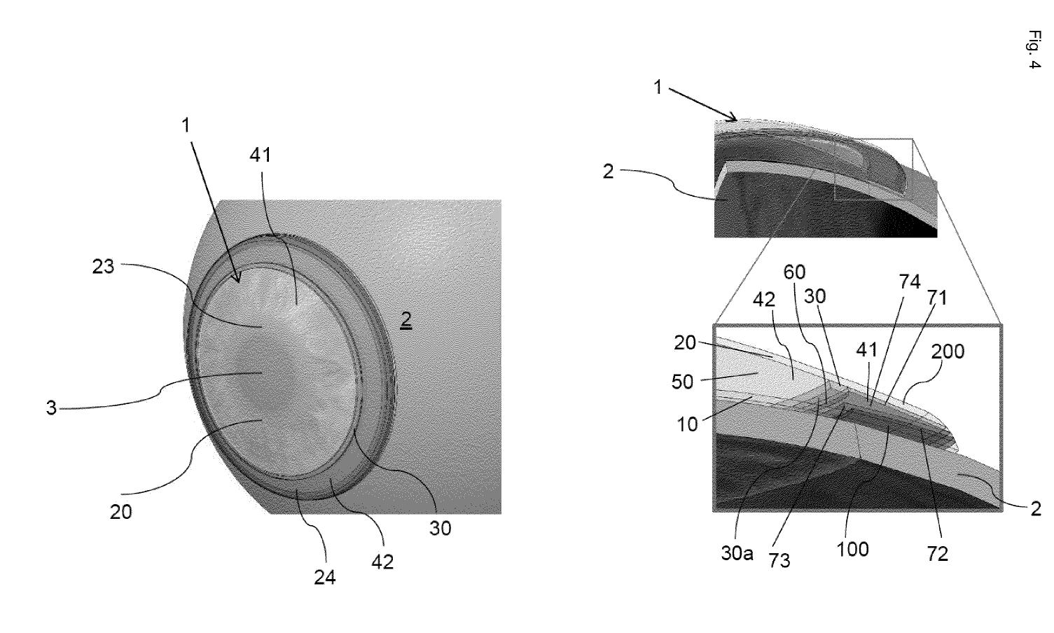

FIG. 5 shows a schematical cross sectional views of the actuator shown in FIG. 4;

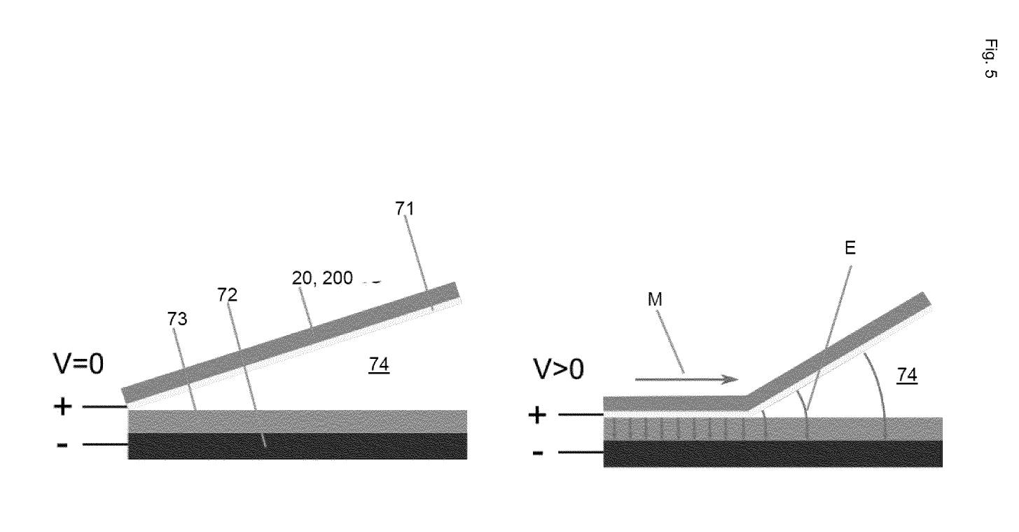

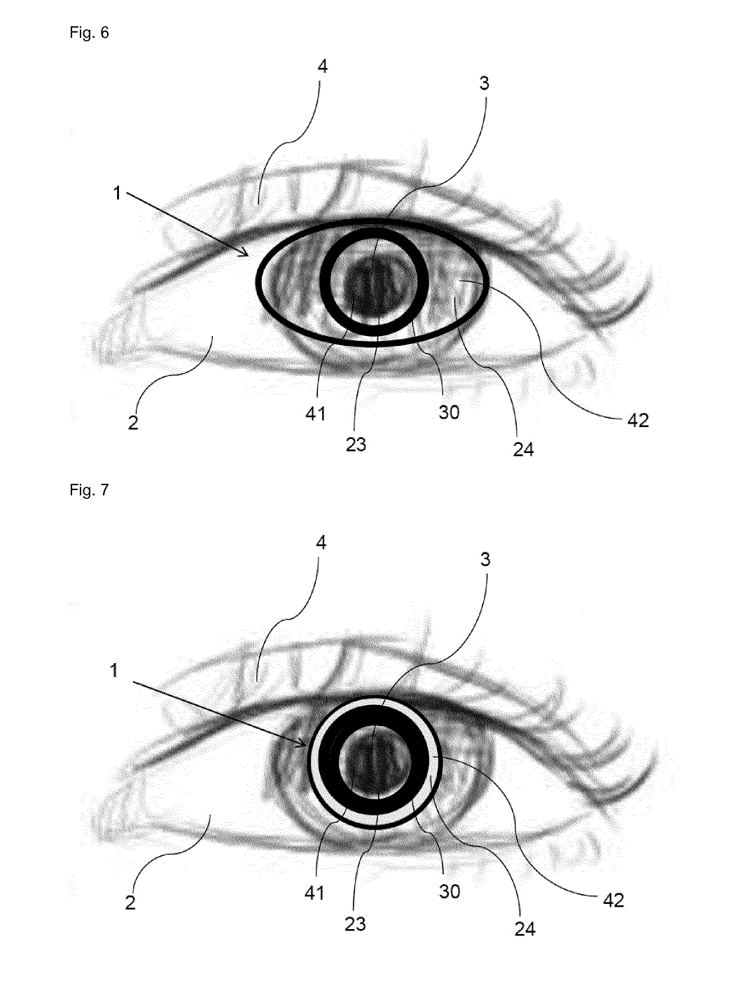

FIGS. 6 to 12 show further embodiments of contact lenses according to the present invention;

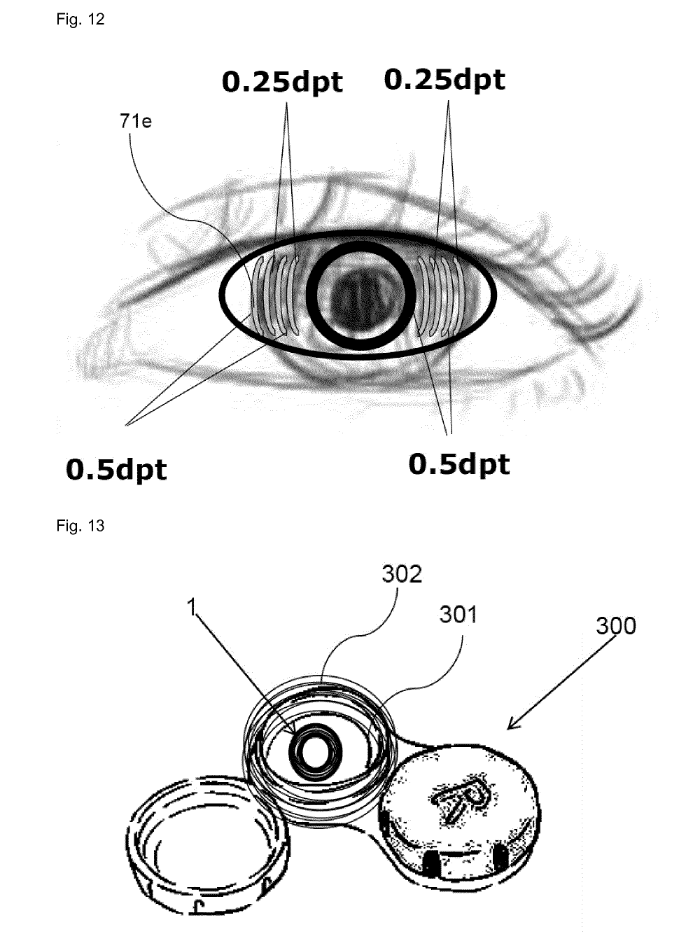

FIG. 13 shows a means for charging a battery of a contact lens according to the invention;

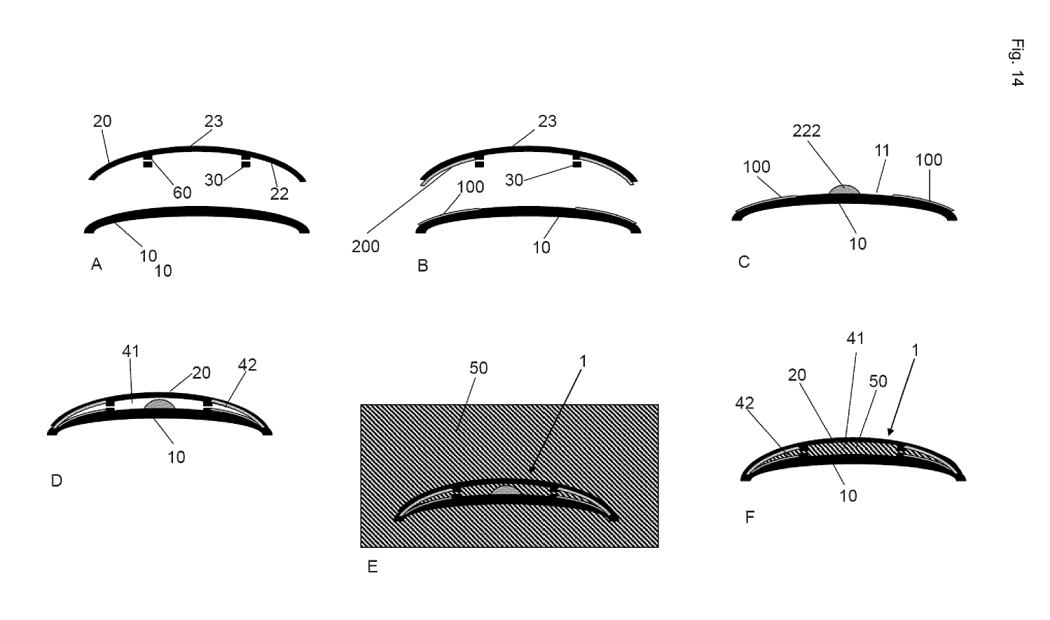

FIG. 14 schematically shows a method for manufacturing a contact lens according to the invention

FIG. 15 shows an alternative method for manufacturing a contact lens according to the invention;

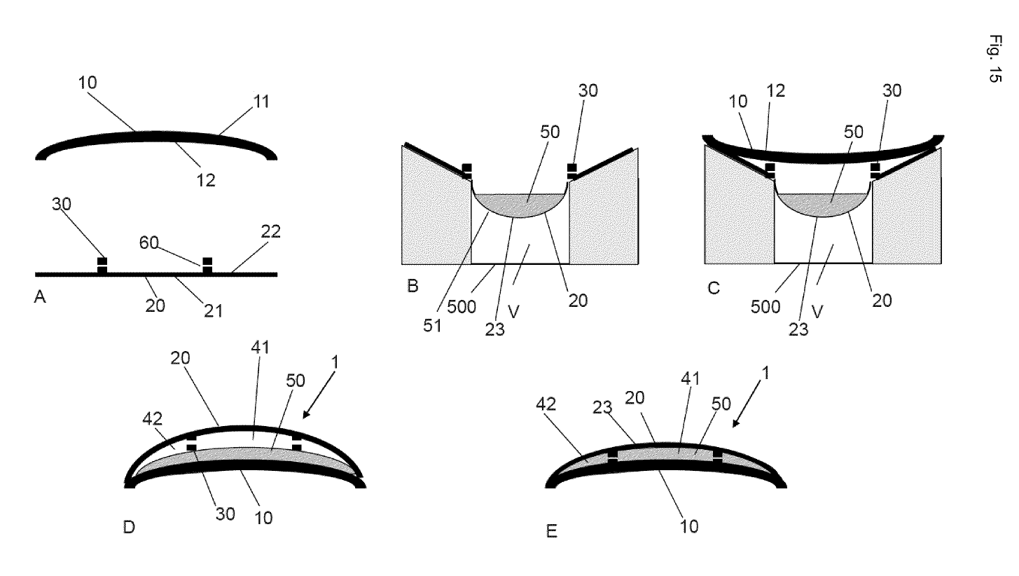

FIG. 16A illustrates low pass filtering of eye blinking movements;

FIGS. 16B illustrates tuning of the time constant of the low pass filtering of FIG. 16A

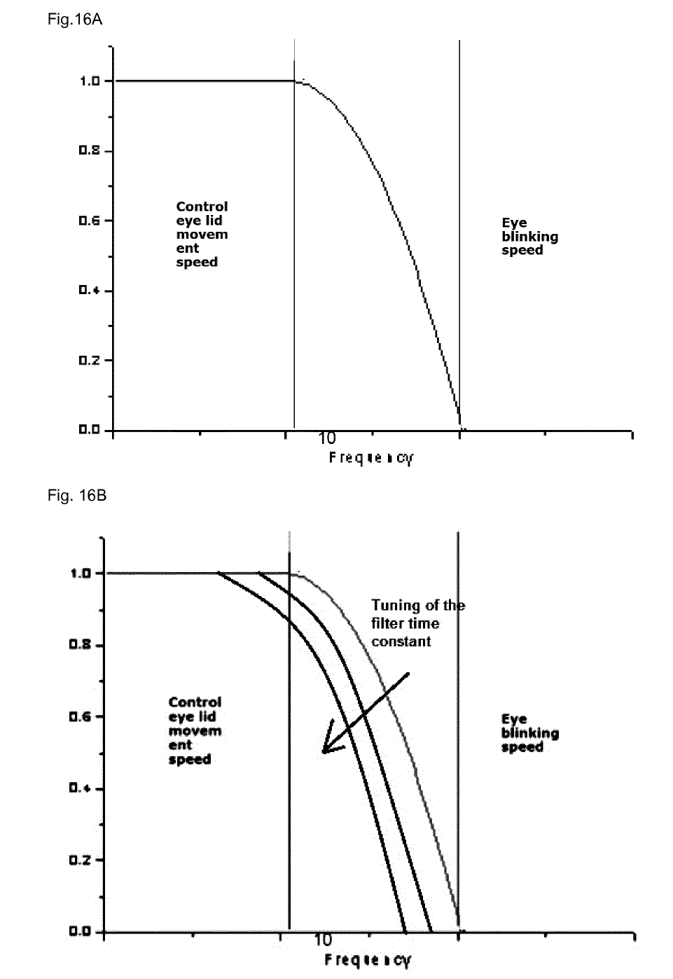

FIG. 17A illustrates an interaction between a contact lens according to the invention and its sensor, actuator, regulator, and processing unit;

FIG. 17B illustrates an interaction between a contact lens according to the invention and its sensor, actuator, regulator, and processing unit;

FIG. 18 shows lenses according to the invention in form of intraocular lenses;

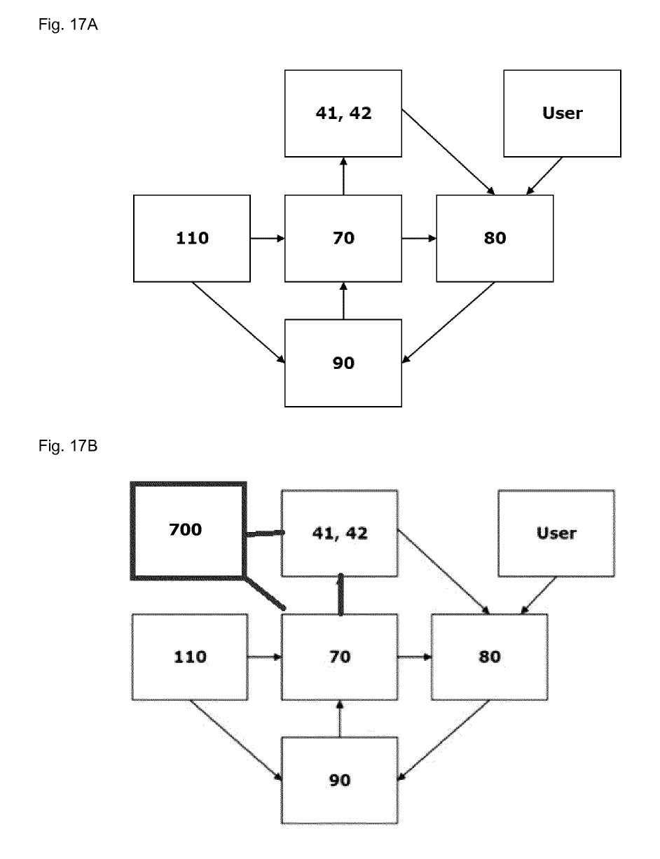

FIG. 19 shows different operation modes, namely when using an active zipper pump (mode A), an active eye lid pump using passive sealing of zipper areas (mode B), or an active eye lid pump using a regulating valve or a frequency control;

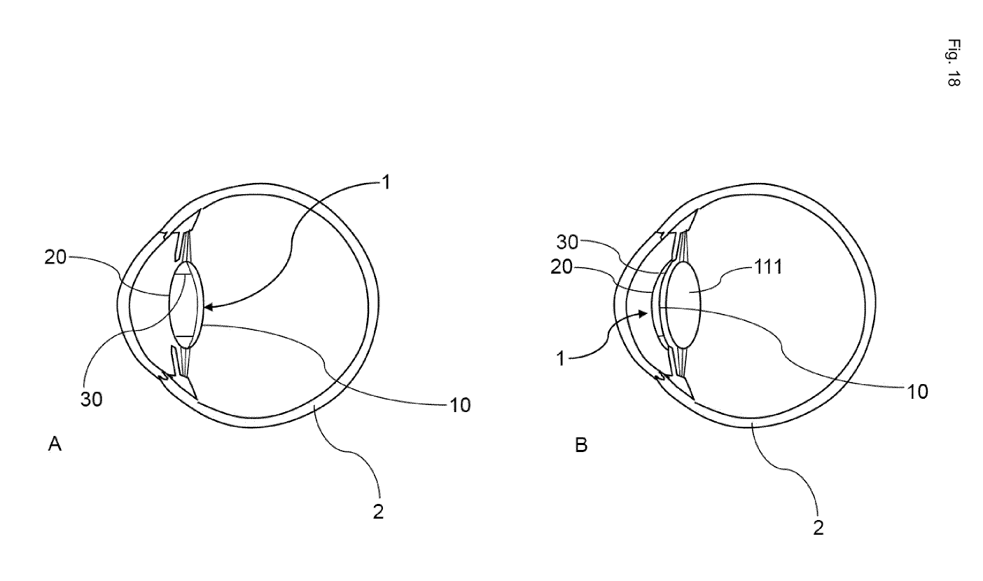

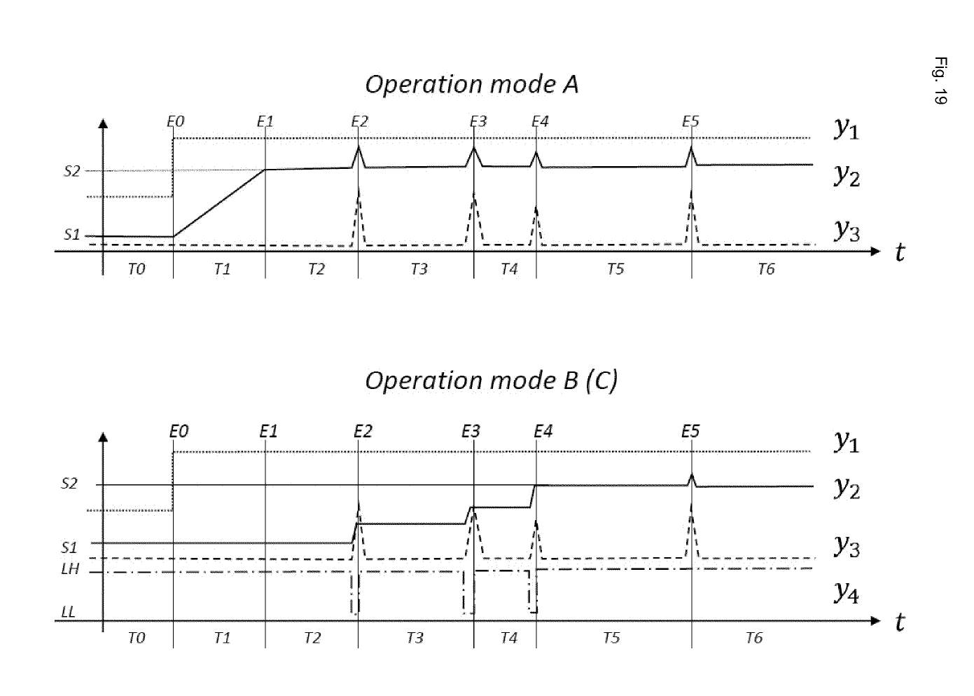

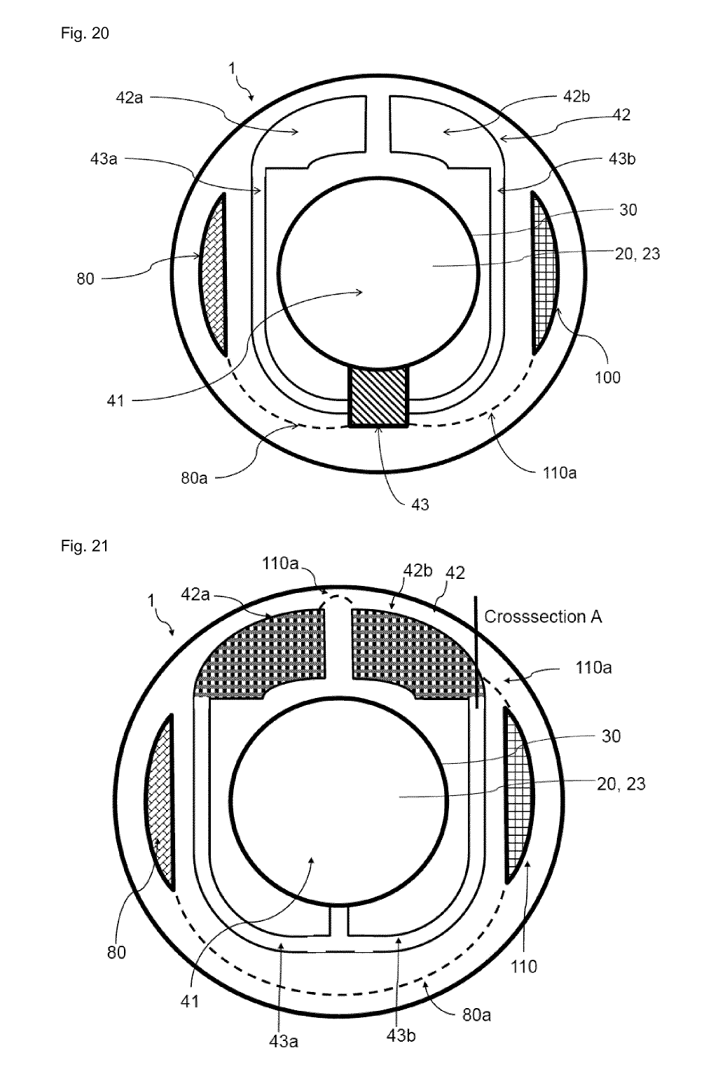

FIG. 20 shows an embodiment of a lens according to the invention using eyelid actuation for changing the focal length of the lens;

FIG. 21 shows a modification of the embodiment shown in FIG. 20;

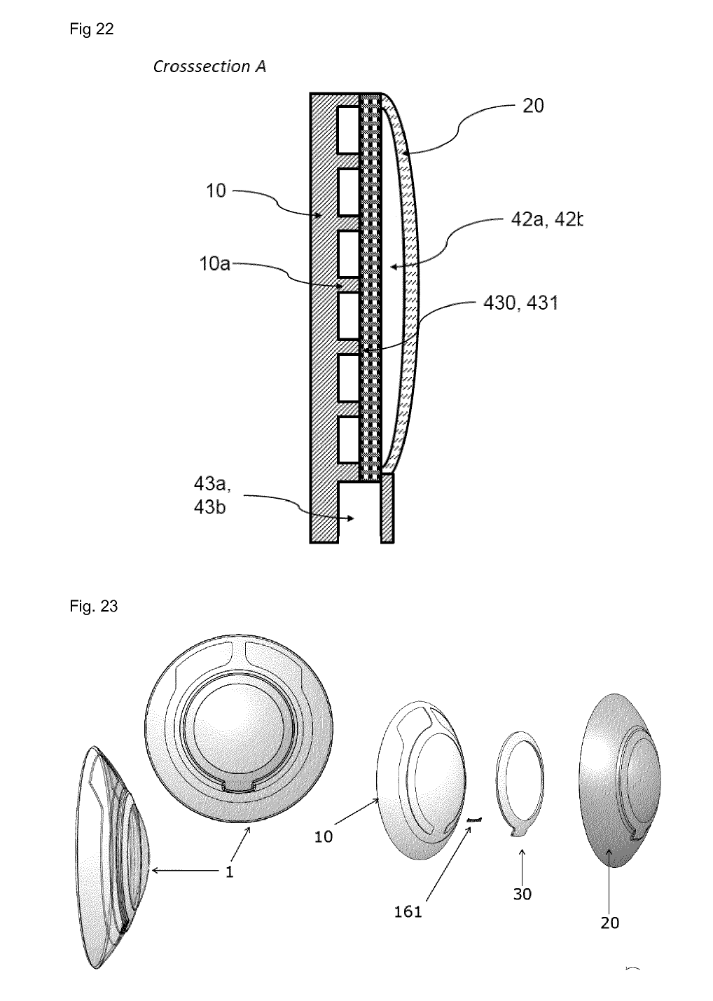

FIG. 22 shows a cross sectional view of the lens of FIG. 21;

FIG. 23-26 shows views of a further embodiment of the lens according to the invention using eyelid actuation for changing the curvature/focal length of the lens;

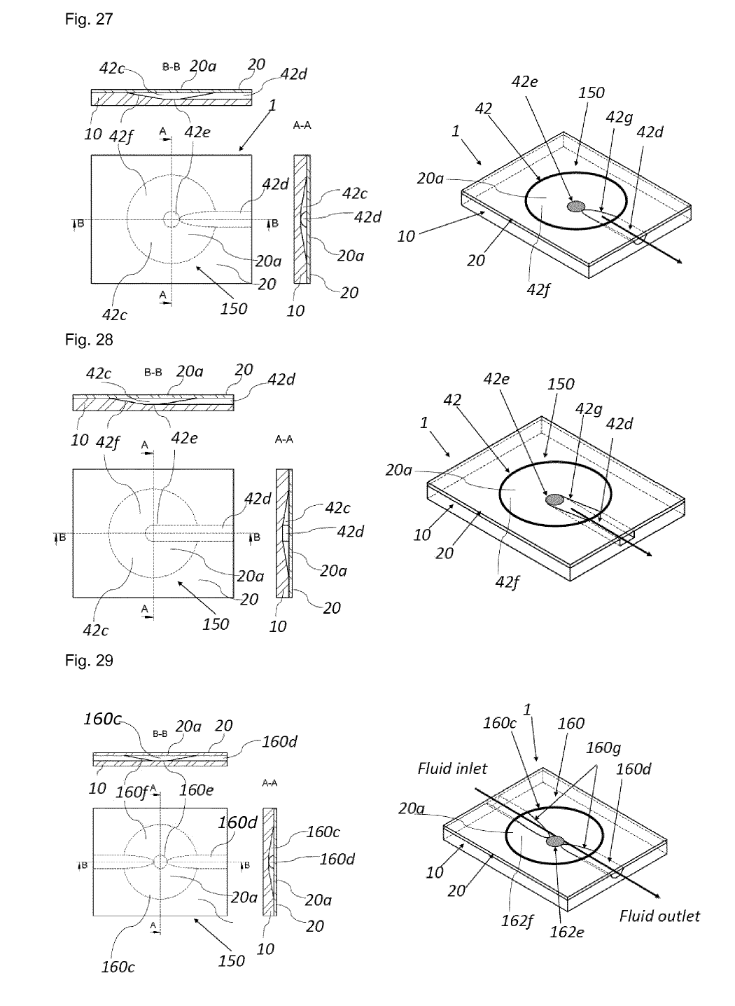

FIG. 27-29 shows different embodiments of lenses with pumps and valves;

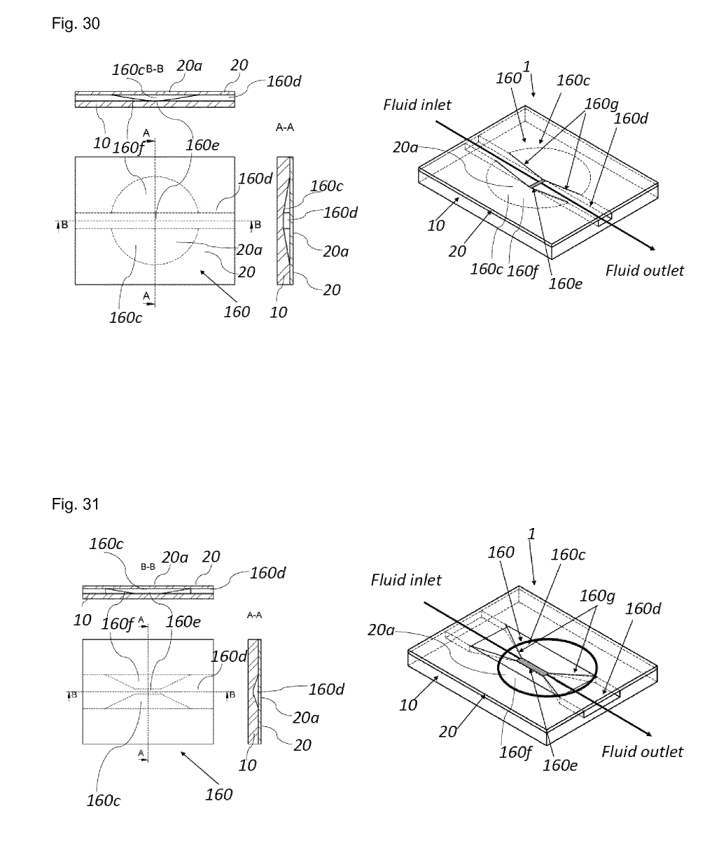

FIG. 30-31 shows different embodiments of lenses with channels and valves;

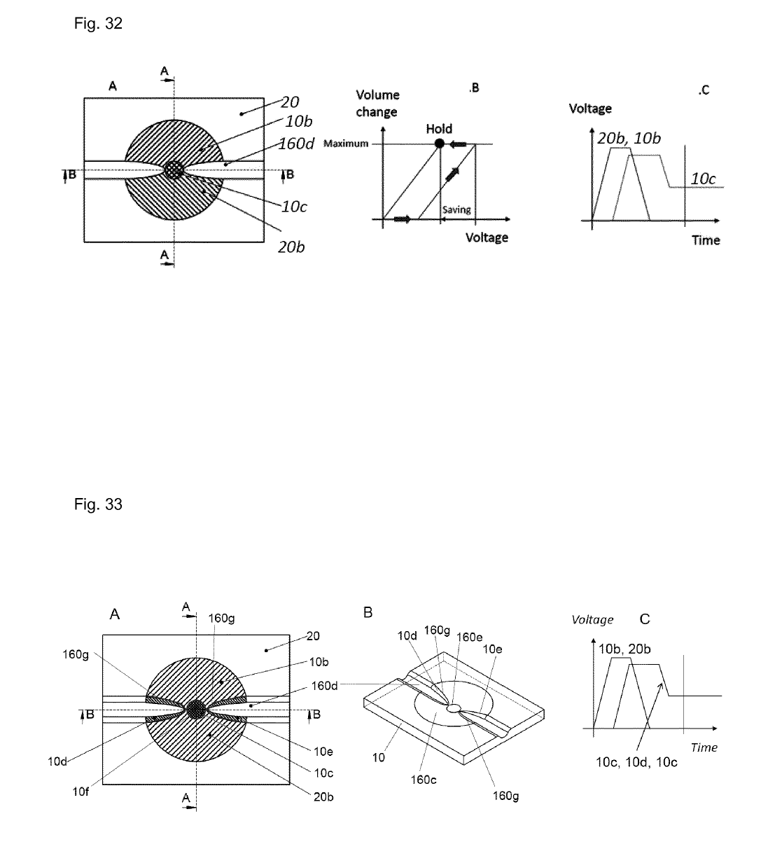

FIG. 32 shows an example of operating electrodes for actuating pumps or valves;

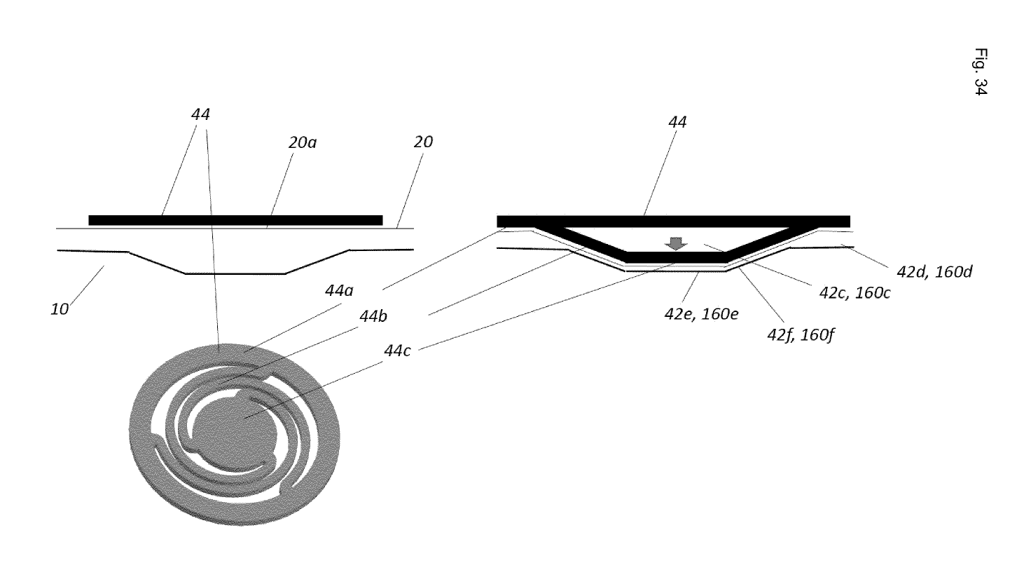

FIG. 33 shows a further example of operating electrodes for actuating pumps or valves;

FIG. 34 shows a valve or pump that is actuated by a member formed out of a shape memory alloy; and

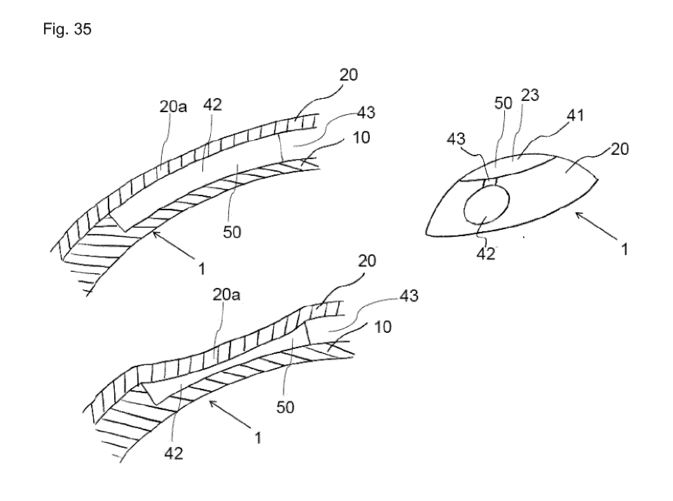

FIG. 35 shows an embodiment of a lens according to the invention using a reservoir that is covered by a bistable membrane region.

FIG. 1 shows an embodiment of a contact lens according to the invention that is designed to be actuated by means of an eyelid 4 of the person wearing the contact lens on the eye 2 associated to the eyelid used for actuating the contact lens. By means of this actuation, the focal length of the contact lens can be adjusted.

In the following the lens may always also be formed as an intraocular lens as shown in FIG. 18 although here, an actuator 70 according to the invention will be particularly used in order to adjust the focal length of such an intraocular lens. The intraocular lens can be e.g. configured to replace the lens of an eye (shown in panel A of FIG. 18) or can be configured to be implanted in addition to the natural lens 111 of the eye 2 as shown in panel B of FIG. 18. The general design of the intraocular lens corresponds to that of a contact lens according to the invention. Further, an intraocular lens according to the invention may comprise an additional fastening means for fastening its position within the eye 2. In the following contact lenses according to the invention are described keeping in mind that these embodiments may also apply in the case of an intraocular lens.

As shown in FIG. 1, the contact lens 1 comprises a base element 10 comprising a back side 12 that is adapted to be arranged on a pupil of a person. The base element 10 further comprises a front side 11 facing away from the back side 12 of the base element 10.

Furthermore, a transparent and elastically expandable membrane 20 is connected to said base element 10, wherein said membrane 20 comprises a back side 22 that faces said front side 11 of the base element 10.

For defining the shape of the deflected membrane 20, particularly of a curvature-adjustable (e.g. central) area 23 of the membrane 20, an e.g. circular ring member 30 is provided (also denoted as lens shaper) that is connected to the back side 22 of the membrane 20 and thus defines said (e.g. circular) area 23 of the membrane 20.

Particularly, the ring member 30 extends circumferentially about the optical axis (indicated by the dashed lines in FIG. 1).

Below this area 23, the contact lens 1 a so-called lens volume 41 which is surrounded by the ring member 30. Further the contact lens 1 comprises a reservoir volume 42 below a boundary area 24 of said membrane 20. These two volumes 41, 42 of the contact lens 1 are filled with the same transparent liquid 50.

In order to be able to adjust the curvature of the curvature-adjustable area 23 of the membrane 22, which area 23 forms a convex bulge in FIG. 1, said volumes 41, 42 are fluidly connected or fluidly connectable to each other such that, when the reservoir volume 42 is compressed, liquid 50 residing in the reservoir volume 42 is pressed into the lens volume 41 such that the curvature of said curvature-adjustable area 23 of the membrane 20 increases and the focal length of the contact lens 1 decreases, and wherein, when the lens volume 41 is compressed, liquid 50 residing in the lens volume 41 is pressed into the reservoir volume 42 such that the curvature of said curvature-adjustable area 23 of the membrane 20 decreases and the focal length of the contact lens 1 increases.

As can be inferred from FIG. 1, the reservoir volume 42 is arranged outside of the ring member 30 in a radial direction (i.e. on an outside of the ring member 30).

In order to actuate a change in curvature of the curvature-adjustable area 23, i.e. in the focal power of the contact lens 1, the reservoir volume 42 is configured to be compressed by an eyelid 4 of an eye 2 of the person when the contact lens 1 is arranged on the pupil 3 of said eye 2 as intended, wherein the reservoir volume 42 is arranged such in the contact lens 1 that the reservoir volume 42 is compressed and the curvature of the curvature-adjustable area 23 of the membrane 20 increases, when said person closes said eyelid 4 partially as shown in FIG. 1 on the right side. Particularly, due to the eyelid 4 sliding onto the boundary region 24 of the membrane 20, the reservoir volume 42 residing below this area 24 is compressed and a corresponding amount of liquid 50 is squeezed into the lens volume 41 leading to an increased curvature of the central area 23 of the membrane 20.

A sequence A to E of such an actuation is shown in FIG. 2, wherein drawing D shows a closing movement of the eyelid 4, where the latter slides onto the central area 23 of the membrane and pushes liquid 50 back into the reservoir volume 42 as shown in panel E.

Preferably, in this embodiment (as shown in FIG. 1 on the left side) the reservoir volume 42 is delimited by a first surface 200 formed by the membrane 20 and by a second surface 100 formed by the base element 10, wherein said surfaces 200, 100 face each other and are configured to stick to each other (e.g. through stiction forces such as van der Waals forces) when making contact upon compression of the reservoir volume 42 such that a compressed state of the reservoir volume 42 can be maintained as indicated e.g. in panel C of FIG. 2. This stiction can be overcome by compressing the lens volume with an eyelid 4 as shown in panel D of FIG. 2.

FIG. 3 shows three different possibilities of establishing a flow connection between the two volumes 41, 42.

According to FIG. 3 (A) the reservoir volume 42 can be fluidly connected to the lens volume 41 via at least one or several openings 60 in the form of channels that reach trough the ring member i.e. extend from an outside of the ring member 30 to an inside of the ring member 30 facing the lens volume 41. Here, the ring member 30 is also connected to the front side 11 of the base element 10.

Alternatively, as shown in FIG. 3 (B), the at least one opening 60 can also be circumferential opening (gap) defined by a face side 30a of the ring member 30 and the front side of the base element 10, wherein said face side 30a faces the front side 11 of the base element 10. Particularly, when the curvature-adjustable area 23 of the membrane 20 assumes a maximal convex curvature, said face side 30a of the ring member 30 may contact the front side 11 of the base element 10. Alternatively, as shown in FIG. 3 (C) the ring member 30 may be attached to the membrane 20 and the base element 10 and comprises recesses formed in its face side 30a which form (e.g. radial) openings or channels 60 extending from the lens volume 41 to the reservoir volume 42. Here, these channels are delimited by the ring member 30 and the front side 11 of the base element 10. In such an embodiment, the ring member 30 may look like a viaduct.

Further, as illustrated in FIG. 16A, the dimensions of the at least one opening 60 or said plurality of openings 60 described above are chosen such that a time period over which the reservoir volume 42 and/or the lens volume 41 have to be compressed in order to yield a change of the curvature of the curvature-adjustable area 23 of the membrane (20) is significantly longer than a typical eye blinking. Thus eye blinking that occurs unwanted will not change the focal power of the contact lens 1.

Further, as illustrated in FIGS. 20-24 and 35, at least one channel 43 or said plurality of channels 43 are used to connect the lens volume 41 to the reservoir volume 42. The at least one opening 60 or plurality of openings 60 are thus connected to the reservoir volume 42 and/or the actuator outlet 160d either directly or indirectly via one or multiple of said channels 43. Here, the opening 60 can also be a channel similar to element 43, and the channel 43 can also include an opening similar to element 60.

Further, as shown in FIG. 16B the low-pass filter time constant can be tuned, e.g. by tuning the cross-sections of the opening 60 or channel 43 (e.g. by means of electrostatic closing).

Here, narrowing the cross section of the at least one opening 60 or channel 43, said plurality of openings 60 or channels 43 described above, can be used to block low frequencies and/or DC components, e.g. the opening 60 or channel 43 could be used as a valve device. This intends to passively (cf. FIG. 16A) or actively (cf. FIG. 16B) reduce the fluid back leakage from the lens volume 41 to the reservoir volume 42). For instance, back leakage may be reduced using a small hole/opening (with non-tunable cross section) (cf. FIG. 16A). Further, back leakage may also be reduced because of a small hole/opening having a tunable cross section (cf. FIG. 16B).

High frequencies are also allowed to pass if the cross section is large enough. Then, eye blinking (see y3 in FIG. 19A and B) can be used as a pulsed pumping source (cf. FIG. 19B). In this case, block 70 below is a mechanic eye lid actuator, which provides the force and energy to power up the lens. The zipper (block 700) assists the eye lid actuator, by adding significant, little, or no pumping power to the power from the mechanical pump 70. Here, assists could also mean that the zipper (block 700) assists the pumping as a passive and/or active regulating device, e.g. by holding the zipper in its closed state after the two electrodes of the zipper have been mechanically (e.g. by eye lid movement) brought to close proximity. It is merely advantageous, to power the zipper already before the electrodes are brought to close or closer proximity (by e.g. the subsequent eye blink) (see y1 in FIG. 19).

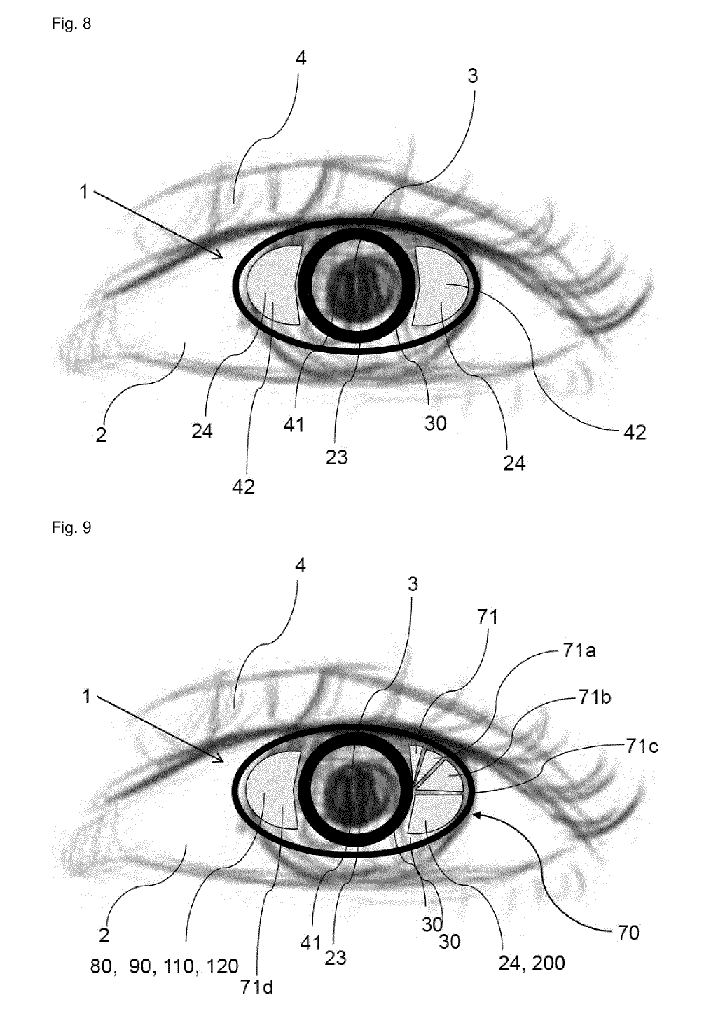

Further, FIGS. 6 and 7 show different possible configurations of the reservoir volume. According to FIG. 6, the contact lens may have an oval contour with a central lens volume 41, wherein here the reservoir volume 42 can be arranged around the lens volume 41 and then as larger portions on either side of the lens volume 41 in the horizontal direction. Further, as shown in FIG. 7 the contact lens 1 may have a circular contour with a circular central lens volume 41 arranged over the pupil 3 of the user and a circular ring-shaped reservoir volume 42 extending around the lens volume 41. Further, as shown in FIG. 8, the reservoir volumes 42 may be located only on the two sides of the lens volume 41.

As an alternative to a powerless actuation of the contact lens 1, the contact lens 1 may comprises at least one actuator 70 that is configured to compress the reservoir volume 42 so as to press liquid 50 from the reservoir volume 42 into the lens volume 41. Again, this actuation may be undone by the eyelid movement shown in FIG. 2, panel D described above.

Such an actuator 70 may be actuated/controlled as indicated in FIG. 17A. According thereto, the contact lens 1 comprises a sensor 80 configured to sense a movement of the person (user) wearing the contact lens 1, and to provide an output signal in response to a pre-determined movement of said person that is made accessible to a processing unit 90. Particularly said movement is a movement of an eyelid 4 of an eye 2 of said user that wears the contact lens 1. The processing unit 90 is configured to actuate the at least one actuator 70 in response to the output signal provided by the sensor 80 in order to transfer liquid from the reservoir volume 42 to the lens volume 41 or vice versa. Further, an electrical energy source 110 is arranged in the contact lens 1 that provides the necessary power for the components 70, 80, 90.

Particularly, the sensor 80 is one of: a photosensitive element, a pressure sensing element, a capacitive sensing element, a thermal sensor, particularly a resistor. For instance, a photosensitive element is arranged such in the contact lens that it can be covered by an eyelid and may thus generate a signal that can be used to control the processing unit 90. The resistor can be used to determine a position of the eyelid 4 since it is sensitive to heat that will be transferred from the eyelid 4 to the resistor. For instance, the resistor can extend along a periphery of the contact lens 1.

Further, the electric energy source 110 can be a battery that can be charged in a variety of different ways, already described above, for instance by means of inductive charging as indicated in FIG. 13. Here, the battery 110 is charged while it rests in a container 300 for the contact lens 1 which comprises a coil 302 connected to a power source which transfers energy to a coil 301 of the contact lens 1 that may extend along the periphery of the contact lens 1.

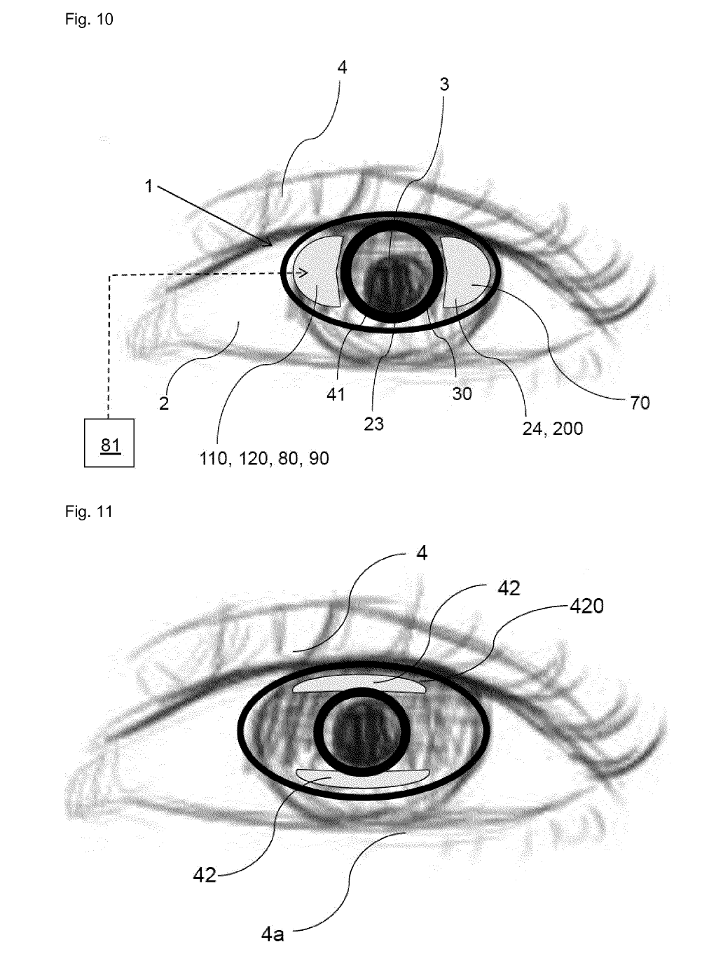

Further, a solar cell 120 may be used in order to charge the battery 110, which solar cell can be arranged, like the battery 110, besides the lens volume 41 outside the ring member 30 as shown in FIGS. 9 and 10, for instance.

Further, the sensor 80 can also sense the status of the contact lens by for example measuring a capacitance of the actuator 70. This can be done by superimposing a high frequency sensing signal to the actuator signal. The sensing signal allows to measure the capacitance of the actuator.

Further, additionally, as shown in FIG. 17B a fluidic device 700 may be added to the embodiment of FIG. 17A (e.g. as a separate block 700), which may be an active regulator and/or passive valve. Alternatively, the actuator unit 70 may be configured to include also passive control features. Besides zippers 70, all other actuators described herein may be used. In particular, also the eye lid blinking itself could be used as an actuator/actuating force, wherein the zipper may only be the regulating device 700. Intentionally, block 700 may be designed to require 1000, or 100, or 10, or at least 2-times less energy and/or (average or peak) power than block 70.

An embodiment of an actuator 70 that can be controlled and powered as described above is shown in FIGS. 4 and 5.

According thereto, the contact lens 1, which may be particularly designed as shown in FIGS. 1 and 3 (right hand side), has a reservoir volume 42 that delimited by a first surface 200 formed by the membrane 20 and a second surface 100 formed by the base element 10, wherein the two surfaces 200, 100 face each other, and wherein the actuator 70 comprises an electrode 71 attached to said first surface 200 and an insulated 73 electrode 72 attached to said second surface 100 such that a tapered gap 74 is formed between the electrodes 71, 72, wherein. Now, in case a voltage is applied by the processing unit 90 to said electrodes 71, 72 as indicated in FIG. 5 said gap 74 is reduced by an amount depending on the magnitude of the applied voltage and liquid 50 is pressed from the reservoir volume 42 into the lens volume 41 which increases the curvature of the curvature-adjustable area 23 of the membrane 20. According to FIG. 9 and FIG. 12 several such actuators 70 having first electrodes 71, 71a, 71b, 71c, 71d and corresponding second electrodes or electrode (not shown since covered by the first electrodes) can be provided on either side of the central lens volume 41 so that a discrete change in curvature of the membrane 20 can be achieved by actuating individual actuator segments (e.g. 71e in FIG. 12). It is for example possible to avoid a continuous adjustment of the actuator by fully closing or opening individual actuator segments. Closing one actuator segment 71e results in a refractive power change of 0.25 dpt or 0.5 dpt. By powering different combinations of actuator segments a broad range of focal length combinations are achievable. These discrete changes may be triggered by certain movement pattern (e.g. of the eyelid 4 of the user) that can be processed accordingly by the processing unit 90.

As further shown in FIG. 10 one or several actuators 70 may only be arranged on one side of the lens volume 41 leaving space for other components such as a battery 110, a solar cell 120, a sensor 80 and a processing unit 90 on the other side of the lens volume 41. Alternatively it is also possible to stack the actuator 70 and the battery 110 or other components on top of each other.

Further, FIG. 10 also indicates that the processing unit 90 may also be configured to actuate the at least one actuator 70 in response to the output signal provided by an external device 81 (e.g. a smart phone). Such an external device may also be used in conjunction with other embodiments of the present invention.

Further, FIG. 11 shows an embodiment in which the reservoir volume 42 is located on the side of the contact lens 1 on which the upper 4 and lower eyelid 4a are located. This allows to push on the reservoir volume without touching the curvature-adjustable area 23 of the membrane, when adjusting the lens curvature with the eyelid.

It is also within the spirit of this invention to have combinations of the discussed embodiments. For example the lens can be adjusted by mechanical pressure via eye lid and the electrostatic actuator is only required to maintain the adjusted curvature of the lens by attracting the boundary area 24 of said membrane 20 to the second surface 100 formed by the base element 10. Alternatively is is also possible to have an insulation layer on the electrode 71 but not on electrode 72. Furthermore it is possible to have the membrane 20 to be the surface in direct contact with the eye and the base element to face the outside world. Furthermore all contact lenses can be embedded in a hydrophilic encapsulation layer. Materials and manufacturing methods as suggested in the following hold for all embodiments described in the FIGS. 1 to 18.

The electrodes 71 (71a to 71d, 71e) and 72 preferably are deformable without being damaged. Advantageously, the first electrodes are therefore manufactured from one of the following materials:
Carbon nanotubes (see “Self-clearable carbon nanotube electrodes for improved performance of dielectric elastomer actuators”, Wei Yuan et al, Proc. SPIE, Vol. 6927, 69270P (2008));
Silver nanowires;
Carbon black (see “Low voltage, highly unable diffraction grating based on dielectric elastomer actuators”, M. Aschwanden et al., Proc. SPIE, Vol. 6524, 65241N (2007));
Carbon grease/conducting greases;
Metal ions (Au, Cu, Cr, . . . ) (see “Mechanical properties of electroactive polymer microactuators with ion-implanted electrodes”, S. Rosset et al., Proc. SPIE, Vol. 6524, 652410 (2007));
Liquid metals (e.g. Galinstan);
Ionic liquids
Electrolytes
Metallic powders, in particular metallic nanoparticles (Gold, silver, copper);
Metal films
Conducting polymers (intrinsically conducting or composites);

The electrodes 71 and 72 may be deposited by means of any of the following techniques:
Spraying;
Ion-implantation (see “Mechanical properties of electroactive polymer microactuators with ion-implanted electrodes”, S. Rosset, Proc. SPIE, Vol. 6524, 652410 (2007));
PVD, CVD;
Evaporation;
Sputtering;
Photolithography;
Printing, in particular contact printing, inkjet printing, laser printing, and screen printing;
Field-guided self-assembly (see e.g. “Local surface charges direct the deposition of carbon nanotubes and fullerenes into nanoscale patterns”, L. Seemann, A. Stemmer, and N. Naujoks, Nano Letters 7, 10, 3007-3012, 2007);
Brushing;
Electrode plating;

To control the stiction behavior of the membrane 20 and the base element 10 the following modifications (e.g. coatings) can be applied to the membrane 20, base element 10, electrodes 71, 72 or insulation layer 73:
Self assembled monolayer (e.g. HMDS)
Fluorocarbons (e.g. perfluorocarbons such as PTFE)
The self assembled monolayer (SAM) can, e.g., comprise molecules with
   Molecule tail groups comprising or consisting of regular or perfluorinated alkyl chains and/or
   Molecule head groups comprising or consisting of silane or phosphoric acid.
Surface topology adjustment by nano-structuring
Surface roughening and/or surface energy modification by e.g.
   nano-structuring
   light (e.g. UV) irradiation
   exposure to ozon and/or other radical gas environments
   ion bombardments

The insulation layer 73 can, e.g., comprise or consist of:
Al2O3, SiO2, Si3N4
Parylene
Epoxy, PVDF (Poly Vinylidene diFluoride)
Electric resins: SU-8, Cyclotene (BCB based),
High-k dielectrics (e.g. inorganic materials, TiO2, HfO2 or ZrO2)
Nanocomposites consisting of high-k nanoparticles (e.g. BaTiO3) in a polymer matrix.
Low-k dielectrics (e.g. polymers)
   CYTOP™ and/or other
   Amorphous polymers and or other
   Fluorocarbon polymers
Cross-linkable polymer dielectrics (e.g.)
Electro-chemical double layers (based on e.g. ionic liquid and ionic gels)

The insulation layer 73 can, e.g., be deposited by means of any of the following techniques:
PVD (Evaporation, sputtering)
CVD (ALD, PECVD, . . . )
Spin-coating
Anodization
Spray pyrolysis

According to an embodiment, the above described actuator 70 using electrodes can be configured to form an active pump which is herein also denoted as active zipper pump which can be configured to be operated in the mode A shown in FIG. 19, wherein y1 denotes a power line, y2 a focal power, y3 the Eye-lid blinks, y4 a control line, E#=the individual event, T#=the respective time interval, S# the focal power state, and wherein LH denotes Logic high, and LL denotes Logic low,

In this mode A, a voltage step at E0 on the power line y1 initiates a period T1 in which the focal power of the lens increases from state S1 to S2. T1 is the zipping duration in which liquid is slowly transferred from the reservoir into the lens volume, e.g, by means of the zipper actuator 70 described above. The focal power change S2-S1 is either defined by zipping to a certain voltage dependent position, or by fully zipping one of many individual actuator segments (e.g. pairs of first and second electrodes 71, 71a, 71b, 71c, 71d, 71e, see above). The energy to transfer the transparent liquid 50 of the lens 1 is extracted from an energy source. In the event of an eye blink, no liquid 50 is permanently transferred, i.e. the focal power prior to the blink event is restored after the blink. The blinking induced focal power variations are small, because no significant liquid volume is transferred during a short blink. Liquid 50 is allowed to only flow slowly in all periods, i.e. the input signal y3 (and also y1) are low-pass filtered and cause slow change in focal power y2.

Alternatively, according to another embodiment, the lens 1 may be operated in the mode B shown in FIG. 19, which corresponds to a lens using an active eye lid pump as well as a passive sealing of zipper areas. Here, passive means that the pumping is e.g. done mechanically, e.g. by pressing onto flexible areas 20a shown in FIGS. 27 to 32, by e.g. using a finger-tip, or as shown in FIG. 35, by flipping a bi-stable element.

Here, a voltage step at E0 alone does not initiate a focal power change. The focal power is incrementally increased at E2, E3, and E4; at which all of the following three causes are true: an eye blinking occurs, the power line y1 is powered, the control line y4 is on high (LH). The energy for the fluid transfer is extracted from the eye lid motion, or from another mechanical source (e.g. pressing with a fingertip or compressing the eye). After setting the control line on low (LL), the focal power is not permanently altered by any eye blink. The liquid transfer during the blinks E2, E3, and E4 is possible, because the liquid's resistance is lower during periods of low control signal. At event E5, significant liquid transfer is not possible due a higher liquid resistance. At E5 less liquid is transferred than at E2,E3, E4.

The control line y4 is not a must. In case of having a control line, the focal power can be freezed anytime at any value by setting the control line to low. In case of not having a control line, the liquid's resistance is permanently low. The focal power will temporarily change at any blinking event. As long as it takes to fully zip one of many individual actuator segments (see above), liquid transfer is permanent, i.e. no or little back flow occurs. After fully closing a segment (e.g. pair of electrodes, see above), blinking only causes a small temporary fluctuation in the focal power, but no permanent change.

Further, alternatively, the lens 1 may be operated in mode C corresponding to an active eye lid pump combined with a regulating valve or a frequency control. Here, the same figure applies as for the mode B, wherein now one has a slow decrease (constant negative slope) of y2 during all time periods.

In case of non-zero back flow, liquid back leakage is compensated i.e. refreshed by subsequent blinks. Continuous focal power states can be addressed depending on the average blinking interval, the liquid flow-in rate, and the liquid back-flow rate (dynamic rate equilibrium). In contrast to mode B, the focal power is set either by controlling the eye blink frequency (user initiated) or by changing the liquid flow resistance (regulating valve for in and/or out flow). A control line y4 is not a must, but can optionally be used to reduce the back-flow rate and/or increase the in-flow rate.

Further FIGS. 14 and 15 show different method for manufacturing a contact lens 1 according to the invention.

Both principle embodiments shown in FIGS. 14 and 15 comprise the steps of: providing a base element 10, providing a transparent and elastically deformable membrane 20 comprising a ring member 30 connected to a back side 22 of the membrane 20, applying coatings (e.g. 200, 100) on the base element 10 and membrane 20 (cf. FIG. 14 A and B and FIG. 15 A and B), bonding the base element 10 to the back side of membrane 20 and thereby forming a lens volume and a reservoir volume of the contact lens (cf. FIG. 14 D and FIG. 15 C), and Filling said lens volume 41 and said reservoir volume 42 with a transparent liquid 50 (cf. FIG. 14 E and FIG. 15 B).

Now, according to FIG. 14, said filling (cf. FIG. 15 E and F) is conducted using osmosis after said bonding has been performed. For this, a pre-defined amount of water soluble salt 222 is arranged on the base element 10 before bonding so that said salt 222 is arranged in the lens volume 41 after bonding, wherein then the bonded base element 10 and membrane 20 is soaked in the transparent liquid 50 which enters the lens volume 41 and reservoir volume 42 by way of diffusion until the osmotic pressure on the inside and outside of the lens 1 is in equilibrium (cf. FIG. 14 F).

As an alternative, according to FIG. 15, said filling (cf. FIG. 15 B and C) is conducted before said bonding, wherein said liquid is filled into a dent 51 formed by the membrane 20, which dent 51 may be formed using a vacuum V acting on the front side 21 of the membrane 20, wherein thereafter said bonding (FIG. 15 C) is conducted, and wherein the lens volume 41 and/or reservoir volume 42 is freed from gas residing therein after said bonding, which is denoted as degassing (cf. FIG. 15 D).

FIG. 20 shows an embodiment of a lens 1 according to the invention that comprises an eyelid actuation. For this, the lens 1 comprises a reservoir volume 42 being filled with the liquid 50 that is positioned in an upper half of the lens 1 (it can also be placed in the lower half for actuation by a lower eyelid), so that the reservoir volume 42 is compressible by an onset of an eyelid movement of an eye of the person when the lens 1 is arranged on the pupil of said eye, so as to pump liquid 50 from the reservoir volume 42 into the lens volume 41 for increasing the curvature of the curvature-adjustable area 23 of the membrane 20 which adjusts the focal power of the lens 1.

As can be seen from FIG. 20, the reservoir volume 42 may comprise two actual reservoirs 42a, 42b arranged in said upper half which are each connectable via a channel 43a, 43b extending along a periphery of the lens volume 41 from the upper half of the lens 1 to the lower half of the lens where they connect to a valve 43 via which liquid can enter the lens volume 41 of the lens 1.

The valve 43 is powered by an energy source 110 that is connected via a power line 110a to the valve 43 and may further be controlled by means of a sensor 80 that is connected to the valve 43 via a data line. For instance, the sensor 80 may detect an eyelid movement that transferred liquid 50 via the channels 43a, 43b into the lens volume through the opened valve 43 and may provide an output signal to close the valve 43 so as to maintain the transferred liquid 50 in the lens volume 41.

Particularly, the valve 43 can be an active or a passive valve system for controlling the in- and out pumping of liquid 50. The (valve) power source preferably requires 1000, or 100, or 10, or at least 2-times less energy than required to tune the lens 1 by means of the membrane 20, 23. The eye lid actuation can also be used to support a pumping system to reduce energy consumption.

Further, in case of passive check-valves, the valve can itself provide the sensor element. The valve energy would be drained from eye-lid pressurized reservoirs.

The valve 43 may be actuated (in case of an active valve 43) by means of
Zipping actuator (e.g. zipping actuator 70 described herein)
Electroosmotic actuation (see below)
EAP (Electro active polymers)
Thin film piezo elements
Electromagnetic-actuator
Shape memory alloy
Phase change material
Thermo-mechanical or bimetallic actuators,
Electrocinetic actuators, or
a magnet that is configured to be moved by a another magnet for opening or closing the valve (e.g. an external magnet, particularly an external magnet that is arranged outside the lens).

Furthermore, the valve 43 can be designed in a way that channels are squeezed by an actuator or clogged or reduced in cross section by any kind of movement of an obstacle to reduce or increase the flow.

Furthermore, in the embodiment shown in FIG. 20 active and passive valve systems may also be combined

For instance in case of a zipping valve 43, channels could be purely passively or actively controlled by means of a zipper device (cross section tuning or complete sealing after every pumping cycle).

Further, the zipping of the device could be assisted by fast blinking pulses (helps to overcome friction and adhesion issues)

FIG. 21 shows in conjunction with FIG. 22 a modification of the embodiment shown in FIG. 20, wherein here each reservoir 42a, 42b comprises its own valve 430, 431 via which the respective reservoir 42a, 42b is connected to its associated channel 43a, 43b, wherein the respective valve 430, 431 comprises an osmotic membrane 430, 431 forming a bottom of the respective reservoir 42a, 42b, which osmotic membrane 430, 431 opens and allows the liquid 50 to pass through it when a suitable voltage is applied to the respective osmotic membrane 430, 431. As shown in FIG. 22, the respective membrane 430, 431 may rest on a support structure 10a formed by the base element 10 which also allows to guide liquid 50 passing the respective membrane 430, 431 into the respective channel 43a, 43b.

In this way the respective osmotic membrane 430, 431 is laying under its associated reservoir 42a, 42b which can be pressurized by the eyelid. Furthermore, the osmotic membranes 430, 431 may be used as current generators by using the reverse electro-osmotic effect.

As before, the lens 1 may further comprise a sensor 80 for detecting an eyelid movement, which sensor 80 is connected to the energy source 110 via a data line 80a, which energy source 110 in turn is electrically connected to said osmotic membranes 430, 431 via corresponding power lines 80a, wherein the sensor 80 is preferably configured to provide an output signal when an eyelid movement is detected by the sensor 80 and to provide the output signal to the energy source 110 which then controls said voltage depending on the output signal.

FIGS. 23 to 26 show a further embodiment of the lens 1 according to the invention, wherein the lens 1 comprises two reservoirs 42a. 42b forming the total reservoir volume 42 of the lens 1, wherein these reservoirs 42a, 42b are each connected via a channel that extends along the periphery of the lens volume 41 to a valve 160 that is arranged in a lower half of the lens 1 so that the lens volume 41 is arranged between the reservoirs 42a, 42b on one side and the valve 160 on the other side. The lens volume is laterally delimited by a ring member 30 that forms a lens shaper to which the membrane 20 is attached so that said curvature-adjustable area 23 of the membrane 20 is defined that covers the lens volume 41 from above.

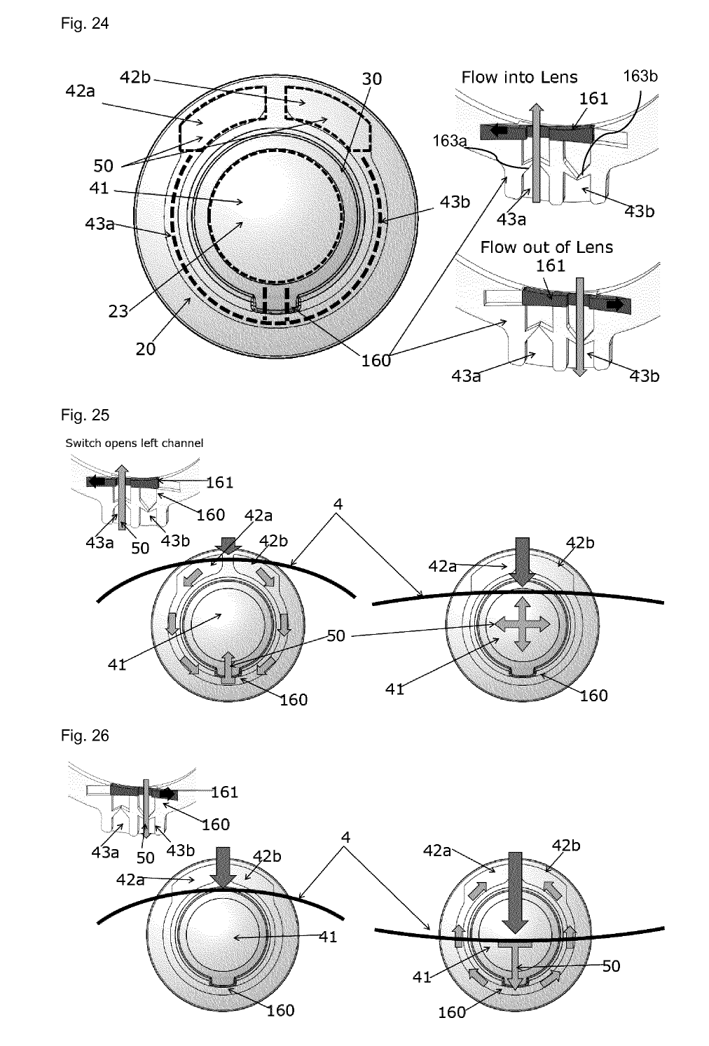

According to FIG. 24 the valve 160 comprises a valve member 163a, 163b for each channel 42a, 42b wherein said two valve members are passive valve members that open (and close) in opposite flow directions as shown in FIG. 24, wherein the valve 160 further comprises a switch 161 that comprises two states, wherein in a first state channel 43a is open and channel 43b is closed and liquid 50 can flow—due to the valve members 163a, 163b from the reservoir volume 42 into the lens volume 41 to decrease the focal length of the lens 1 by increasing the curvature of the area 23 of the membrane 20 of the lens 1.

In the second state channel 43b is open and channel 43a closed, and due to the valve members 163a, 163b liquid 50 can flow out of the lens volume 41 into the reservoir volume 42.

In FIGS. 23 to 26, the liquid flow 50 is actuated by an eyelid 4 of the user as shown in FIGS. 25 to 26. In order to decrease the focal length of the lens 1, liquid is pumped by means of an eyelid movement from the reservoirs 42a, 42b into the lens volume 41 via valve 160 which has its switch in the first state. Once this transfer of liquid 50 is complete (when the eyelid has moved past the reservoirs 42a. 42b as shown on the right hand side of FIG. 25) liquid 50 cannot escape the lens volume due to valve member 163a shown in FIG. 24.

In case liquid 50 shall be pumped out of the lens volume 41 in order to increase the focal length of the lens 1, the switch 161 is moved into its second state shown in FIG. 24 such that liquid 50 can be pushed out of the lens volume 41 into the reservoirs 42a, 42b via valve member 163b by means of the eyelid 4 movement shown in FIG. 26 on the right hand side.

The switch 161 can be actuated using actuators but may also be manually actuated to change the state of the switch 161.

Further, FIG. 27 shows yet another embodiment of a lens 1 according to the invention. Here, the lens 1 comprises a pump 150 which comprises the reservoir volume 42, wherein the pump 150 is configured to empty the reservoir volume 42 by pulling a region 20a of said membrane 20 that covers the reservoir volume 42 into a dent 42c that is formed in the base element 10 and forms part of the reservoir volume 42 in which the transparent liquid 50 of the lens resides,

As shown in FIG. 27 the dent 42c may comprises a concave shape, but may also comprise a conical shape as shown in the embodiment of FIG. 28.

Preferably, the pump 150 is configured to generate an electrostatic force for pulling said region 20a of the membrane 20 into the dent 42c, wherein for generating said force said region 20a of the membrane 20 forms a flexible and particularly stretchable, electrically conducting electrode 20b (see FIG. 32), and the base element 10 forms at least one corresponding counter electrode 10b (see FIG. 32).

As further shown in FIGS. 27 and 28, the dent 42c / reservoir 42 of the lens is connected to the lens volume 41 (not shown here) via a channel 42d that may be formed by a groove in the base element 10. The channel 42d preferably leads to a lowest area 42e of a bottom 42f of said dent 42c of the reservoir volume 42 for draining said dent 42c, wherein said groove/channel 42d is configured to be automatically sealed when said region 20a of the membrane 20 is pulled into the dent 42c by means of said electrodes 10b, 20b (10c, see below),

When said groove/channel 42d is sealed by the pulled-in region 20a, re-entry of liquid 50 into the reservoir volume 42/dent 42c is blocked at an intersection 42g of the groove/channel 42d and the reservoir volume 42, which intersection 42g is also denoted as sealing line and indicated in FIGS. 27 and 28. Please note that the cross section of the channel 42d in FIG. 27 is curved while it is rectangular in FIG. 28 which leads to different geometries of the sealing lines 42g.

Further, the pump 150 is configured to keep the channel 42d in its sealed or closed state by pinning said region 20a of the membrane 20 to an area 42e on the bottom 42f of said dent 42c of the reservoir volume 42 (this area 42e is also denoted as sealing area) using the electrode 20b of the membrane 20 on one side and on the other side said counter electrode 10b and/or a central electrode 10c that is arranged at the center of the bottom 42f of the dent 42c and surrounded by said counter electrode 10b as shown in FIG. 32 (note that FIG. 32 actually shows a combination of a channel 160d and a valve 160 that will be described below, but also applies to the combination of a pump 150 and a valve shown in FIGS. 27 and 28.

The active electrode area and the electric power can be reduced after pinning the membrane 20 to the bottom 42f of the dent 42c /reservoir volume 42. Furthermore. Depending on the voltages applied to said electrode 10b, 20b, 10c, the sealed channel 42d is configured to open at a certain back pressure, which initiates liquid back flow and refilling of the reservoir volume 42.

FIG. 29 shows yet another embodiment of a lens 1 according to the invention, wherein the lens 1 now comprises a channel 160d for providing a flow connection between the reservoir volume 42 and the lens volume 41 (not shown), wherein the lens 1 comprises a valve 160 for opening or closing said channel 160d, wherein said channel 160d extends through a dent 160c (forming an adjustable volume) of the valve 160 formed in the base element 10, which dent 160c is covered by a region 20a of said membrane 20, wherein the valve 160 is configured to open or block said channel 160d by pulling a region 20a of said membrane 20 covering the dent 160c into the dent 160c.

Also here, the valve 160 is configured to generate an electrostatic force for pulling said region 20a of the membrane 20 into the dent 160c of the valve 160 for closing the valve 160/channel 160d, wherein for generating said force said region 20a of the membrane 20 forms a flexible and particularly stretchable, electrically conducting electrode 20b, and the base element 10 forms at least one corresponding counter electrode 10b.

Now, the channel 160d is configured to be automatically blocked when said region 20a of the membrane 20 is pulled into the dent 160c of the valve 160. When said channel 160d is blocked, re-entry of liquid 50 into the dent 160c and through the dent 160c of the valve 160 is blocked at intersections 160g of the channel 160d and the dent 160c which intersections are again denoted as sealing lines and are indicated in FIGS. 29 to 31.

Preferably, the valve 160 is configured to keep the channel 160d in its blocked state by pinning said region 20a of the membrane 20 to an area 160e on the bottom 160f of said dent 160c of the valve 160 (this area is also denoted as sealing area) using the electrode 20b of the membrane 20 on one side and said counter electrode 10b and/or a central electrode 10c that is arranged at the center of the bottom 160f of the dent 160c and surrounded by said counter electrode 10b (cf. FIG. 32).

Again, the active electrode area and the electric power can be reduced after pinning the membrane 20 to the bottom 160f of the dent 160c /reservoir volume 42.

Also here, depending on the electric power applied, the valve 160 is configured to open at a certain pressure, which allows passage of liquid 50 between the reservoir volume 42 and the lens volume 41.

FIGS. 30 to 31 show modifications of the embodiment shown in FIG. 29, wherein in FIGS. 30 and 31 the geometry (cross section) of the channels 160d is different, leading to modified sealing lines 160g.

FIG. 32 illustrates the operation of the electrodes 10b, 20b, 10c in case of the channels and valves shown in FIGS. 29 to 31 (however this operation can also be applied to the actuation of pumps 150 in FIGS. 27 and 28.

According to FIG. 32 A, B and C, in order to keep the valve (i.e. the channel 160d) in its closed/sealed state, it is sufficient to pin the region 20a of the membrane 20 at a small area 42e onto the reservoir bottom 42f (FIG. 32 A, 10c). Here, the central electrode 10c could be electrically isolated from the electrodes 10b, 20b and could be individually addressed.

After deflecting the region 20a of the membrane 20 to the maximum deflection state it touches the base element 10, the voltage applied can then be reduced to save static power during idle times (FIG. 32 B saving).

After activating power on electrode 10c (FIG. 32 C), the voltage on electrodes 10b, 20b can be reduced or completely removed. This helps to lower the static power consumption.

The electrodes 10b, 20b, 10c may consist of different materials and different thicknesses to optimize leakage current and operation voltage. On one hand, the small area electrode 10c could be covered with a thin (e.g. 0.1 to 10 micrometer) or ultra-thin (e.g. smaller than 100 nanometer), high-k, high-dielectric strength, e.g. non-flexible, inorganic dielectric material (e.g. Al₂O₃), to minimize static power consumption. On the other hand, the large area electrode 10b, and 20b could be covered with a thin (e.g. 0.5 to 5 micrometer) or ultra-thin (e.g. smaller than 0.5 micrometer), low-k, high-dielectric strength, flexible inorganic dielectric (e.g. Parylene or PDMS based)

Furthermore, the electrodes 10b, 20b could be fabricated with a radial gradient in the dielectric susceptibility and/or dielectric thickness, such that the local areal capacitance increases towards the center. In this way a larger maximum deflection can be achieved at a given voltage and leakage current.

FIG. 33 illustrates a further example of an operation of the electrodes 10b, 20b, 10c in case of the channels and valves shown in FIGS. 29 to 31 (however this operation can also be applied to the actuation of pumps 150 in FIGS. 27 and 28).

Here, additional sealing line electrodes 10d, 10e may be used which are separated from the central electrode 10 by a gap 10f.

Again, in order to keep the valve 160 (or a pump 150) in its closed state, it is sufficient to pin the membrane 20 at a small area 160g and/or 160e (cf. FIG. 33 B). The electrodes 10c, 10d or 10e can be electrically isolated from the electrodes 10b and from each other, 20b and can further be individually addressed.

To seal the valve 160, it is sufficient to pin the membrane 20 at a small areas following the sealing lines 160g. Ideally, the electrodes 10b, 20b, 10c, 10d, 10e are isolated from each other by a lateral gap 10f.

Said electrodes 10b, 20b, 10c, 10d, 10e may consist of different materials and different thicknesses to optimize leakage current and operation voltage. On one hand, the small area electrodes 10c, 10d, 10e can be covered with an ultra-thin (<1 micrometer), high-k, high-dielectric strength, eventually non-flexible, inorganic dielectric material (e.g. Al₂O₃), to minimize static power consumption. On the other hand, the large area electrode 10b and 20b could be covered with a thin (1-2 micrometer, low-k, high-dielectric strength, flexible inorganic dielectric (e.g. Parylene or PDMS based).

The electrodes 10b, 20b may be fabricated with a radial gradient in the dielectric susceptibility and/or dielectric thickness, such that the local areal capacitance increases towards the center. In this way a larger maximum deflection can be achieved at a given voltage and leakage current.

Further, as shown in FIG. 34, as an alternative to said electrodes 10c, 10b, 20b, and particularly 10d and 10e, the pump 150 or valve 160 described herein may also be actuated using a member 44 that is made out of a shape memory alloy (e.g. such as Nitinol). The member 44 may coupled to said region 20a of the membrane 20 and comprises a first flat state shown on the left hand side of FIG. 34, wherein upon heating said member 44 by means of an electrical current it changes to its expanded state shown on the right hand side of FIG. 34, in which state the member 44 moves (e.g. pushes or pulls) said region 20a of the membrane into the dent 42c, 160c of the pump 150 or valve 160.

Particularly, said member may comprise a circumferential (e.g. annular) frame 44a which is integrally connected to a central plate 44c via elongated curved arms 44b. In the expanded state, the arms 44b expand downwards so that the plate 44c moves said region 20a of the membrane 20 into the dent 42c, 160c and seals the reservoir/valve.

Furthermore, as shown in FIG. 35 an embodiment of a lens according to the invention is disclosed that comprises a reservoir pump mechanism with a bistable membrane region 20a.

Particularly, the reservoir volume 42 is covered by a bistable region 20a of the membrane 20 of the lens 1, wherein said region 20a is movable with respect to the base element 10 from a first stable state to a second stable state and vice versa, wherein in the first state the reservoir volume 42 is larger than in the second state, and wherein when said region 20a is moved from the first state to the second state, liquid 50 flows from the reservoir volume 42 into the lens volume 41, and wherein when the region 20a is moved from the second state to the first state, liquid flows from the lens volume 41 back to the reservoir volume 42.

The lens 1 further comprises a channel 43 that connects the reservoir volume 42 to the lens volume 41 to allow liquid 50 to flow from the lens volume 41 to the reservoir volume 42 and vice versa when the state of the region 20a changes accordingly.

As indicated in FIG. 35, the reservoir volume 42 may comprises a circular shape, but may also comprise a ring shape extending around the lens volume 41.

Said portion 20a of the membrane 20 can be made of metal, or a polymer, or an elastomer, or a heterogeneous structure of at least two materials. For example: a disk of Kapton embedded in silicone.

The use of the lens according to the invention is very versatile and further includes without limitation devices such as: vision systems, ophthalmic lenses (contact lenses and intraocular lenses), ophthalmology equipment such as phoropter, refractometer, fundus cameras, ppt. biometrie, perimeter, refractometer, tonometer, anomaloskop, kontrastometer, endothelmicroscope, anomaloscope, binoptometer, OCT, rodatest, ophthalmoscope, RTA, slitlamp microscope, surgical microscope, auto-refractometer, keratograph, confocal imager, Scheimpflug camera, wavefront aberrometer, pupillometer, skin laser, eye laser, otoscope, laryngoscope, Raman spectrometer, portable spectrometer, photodynamic diagnosis; as well as lighting devices, lighting fixtures, devices for machine vision, laser processing devices, devices for conducting a light show, printers, metrology devices, (e.g. head-worn) glasses, medical devices, robot cams, motion tracking devices, microscopes, telescopes, endoscopes, binoculars, surveillance cameras, automotive devices, projectors, range finder, bar code readers, and web cams, fiber coupling, biometric devices, electronic magnifiers, motion tracking, intra-ocular lenses, mobile phones, military, digital still cameras, web cams, microscopes, telescopes, endoscopes, binoculars, research, industrial applications.

While there are shown and described presently preferred embodiments of the invention, it is to be distinctly understood that the invention is not limited thereto but may be otherwise variously embodied and practiced within the scope of the following claims.

1. A lens (1) for vision correction, wherein the lens (1) is configured to be placed directly on the surface of an eye (2) of a person or to be implanted into an eye (2) of a person, and wherein the lens (1) further comprises:
a transparent base element (10) having a back side (12) and a front side (11) facing away from the back side (12),
a transparent and elastically expandable membrane (20) connected to said base element (10), wherein said membrane (20) comprises a back side (22) that faces said front side (11) of the base element (10),
a ring member (30) connected to said back side (22) of the membrane (20) so that the ring member (30) defines a curvature-adjustable area (23) of the membrane (20), and
wherein the lens (1) comprises a lens volume (41) adjacent said curvature-adjustable area (23) of the membrane (20), which lens volume (41) is delimited by the ring member (30), and wherein the lens (1) comprises a reservoir volume (42) adjacent a boundary area (24) of said membrane (20), wherein said two volumes (41, 42) are filled with a transparent liquid (50), and
wherein said volumes (41, 42) are fluidly connected or fluidly connectable to each other such that, when the reservoir volume (42) is compressed, liquid (50) residing in the reservoir volume (42) is pressed into the lens volume (41) such that the curvature of said curvature-adjustable area (23) of the membrane (22) increases and the focal length of the lens (1) decreases.

2. The lens according to claim 1, characterized in that the lens volume (41) is configured to be compressed, wherein when the lens volume (41) is compressed, liquid (50) residing in the lens volume (41) is pressed into the reservoir volume (42) such that the curvature of said curvature-adjustable area (23) of the membrane (22) decreases and the focal length of the lens (1) increases.

3. The lens according to claim 1, characterized in that the reservoir volume (42) is fluidly connected or fluidly connectable to the lens volume (41) via at least one opening (60).

4. The lens according to claim 3, characterized in that the at least one opening (60) is a circumferential gap defined by a face side (30a) of the ring member (30), which face side (30a) faces the front side (11) of the base element (10), and the base element (10), wherein particularly, when the curvature-adjustable area (23) of the membrane (20) assumes a maximal convex curvature, said face side (30a) of the ring member (30) contacts the front side (11) of the base element (10).

5. The lens according to claim 2, characterized in that the ring member (30) is also connected to the front side (11) of the base element (10), wherein the at least one opening (60) is a channel extending through the ring member (30), wherein particularly the ring member (30) comprises a plurality of openings (60) in the form of channels extending through the ring member (30), which channels fluidly connect the reservoir volume (42) to the lens volume (41).

6. The lens according to claim 2, characterized in that the ring member (30) is also connected to the front side (11) of the base element (10), wherein the at least one opening (60) is a channel delimited by the ring member (30) and the front side (11) of the base element (10).

7. Lens according to claim 3, characterized in that the dimensions of the at least one opening (60) or said plurality of openings (60) are chosen such that a time period over which the reservoir volume (42) and/or the lens volume (41) have to be compressed in order to yield a change of the curvature of the curvature-adjustable area (23) of the membrane (20) is longer than a blink of an eyelid.

8. Lens according to claim 3, characterized in that the dimensions of the at least one opening (60), of the reservoir volume (42) and of the lens volume (41) are selected such that the total amount of liquid (50) that is transferred from the lens volume (41) to the reservoir volume (42) during one complete blink of an eyelid (4) of an eye (2) on which the lens (1) is placed or into which the lens (1) is implanted is smaller than the amount of liquid (50) required to change the optical power of the lens (1) by more than 0.25 diopter, particularly by more than 0.1 diopter, particularly by more than 0.05 diopter.

9. Lens according claim 1, characterized in that the lens volume (41) is configured to be compressed by an eyelid (4) of an eye (2) of the person when the lens (1) is arranged on the pupil (3) of said eye (2), particularly by fully closing said eyelid (4).

10. Lens according to claim 1, characterized in that the reservoir volume (42) is configured to be compressed by an eyelid (4) of an eye (2) of the person when the lens (1) is arranged on the pupil (3) of said eye (2), wherein particularly the reservoir volume (42) is arranged such in the lens (1) that the reservoir volume (42) is compressed and the curvature of the curvature-adjustable area (23) of the membrane (20) increases, when said person closes said eyelid (4) partially.

11. Lens according to claim 1, characterized in that the reservoir volume (42) is delimited by a first surface (200) formed by the membrane (20) and by a second surface (100) formed by the base element (10), wherein said surfaces (200, 100) face each other, and wherein said surfaces (200, 100) are configured to stick to each other when making contact upon compression of the reservoir volume (42) such that a compressed state of the reservoir volume (42) can be maintained without an eyelid (4) pushing onto the reservoir volume (42).

12. Lens according to claim 11, characterized in that the first surface (200) and second surface (100) stick to each other through electrostatic attraction, magnetic attraction or van der Waals forces.

13. Lens according to claim 1, characterized in that the lens (1) comprises at least one actuator (70) that is configured to compress the reservoir volume (42) so as to press liquid (50) from the reservoir volume (42) into the lens volume (41).

14. Lens according to claim 1, characterized in that the reservoir volume (42) is delimited by a first surface (200) formed by the membrane (20) and a second surface (100) formed by the base element (10), wherein the two surfaces (200, 100) face each other.

15. Lens according to claim 13, characterized in that the actuator (70) comprises at least a first electrode (71) attached to said first surface (200) and at least a second electrode (72) attached to said second surface (100) such that a gap (74) is formed between the electrodes (71, 72) that is adjustable in size by means of a voltage applied to the electrodes such that, when the gap is reduced, liquid (50) is pressed from the reservoir volume (42) into the lens volume (41), and wherein, when the voltage applied to said electrodes (71,72) is decreased, a tension of the membrane causes liquid (50) to flow back from the lens volume (41) into the reservoir volume (42).

16. The lens according to claim 13, characterized in that the actuator (70) comprises one or a plurality of first electrodes (71, 71a, 71b, 71c, 7d) attached to said first surface (200) and a corresponding number of second electrodes (72) attached to said second surface (100) such that a pair of a first and a second electrode (71, 72) or pairs of first and second electrodes (71, 71a, 71b, 71c, 7d, 71e, 72) are formed, wherein each pair of electrodes (71, 71a, 71b, 71c, 71d, 71e, 72) delimits an associated gap (74) arranged between the respective first and second electrode (71, 71a, 71b, 71c, 71d, 71e, 72) that is closable by means of a voltage applied to the respective pair of electrodes such that, when the respective gap (74) is closed, liquid (50) is pressed from the reservoir volume (42) into the lens volume (41), and wherein, when the voltage applied to the respective pair of electrodes (71, 71a, 71b, 71c, 71d, 71e, 72) is decreased or turned off, the respective gap (74) opens and a tension of the membrane (20) causes a corresponding amount of liquid (50) to flow back from the lens volume (41) into the reservoir volume (42).

17. The lens according to claim 15, characterized in that the at least one first electrode (71) is electrically insulated with respect to the at least one second electrode (72), or that each first electrode (71, 71a, 71b, 71c, 71d; 71e) is electrically insulated with respect to the associated second electrode (72).

18. The lens according to claim 1, characterized in that the reservoir volume (42) is arranged in a boundary region (420) of the lens (1) so that, when the lens (1) is arranged with respect to an eye (2) as intended, the reservoir volume (42) faces the eyelid (4) of said eye (2) and said eyelid (4) is partially closable such that it only pushes onto the reservoir volume (42) but not on the lens volume (41).

19. The lens according to claim 15, characterized in that for reducing an influence of an eyelid (4) on the reservoir volume (42) and said electrodes (71, 71a, 71b, 71c, 71d, 71e, 72), the reservoir volume (42) is arranged next to the lens volume (41) in a horizontal direction when the lens (1) is arranged with respect to an eye (2) as intended.

20. The lens according to claim 13, characterized in that the at least one actuator (70) extends circumferentially around the ring member (30).

21. The lens according to claim 1, characterized that the ring member (30) is 5 times, particularly 10 times, particularly 50 times, particularly 100 times, particularly 1000 times stiffer than the membrane (20).

22. The lens according to claim 1, characterized in that the ring member (30) has a circularity and flatness better than 25 μm, particularly better than 10 μm, particularly better than 5 μm at an interface between the ring member (30) and the membrane (20).

23. The lens according to claim 13, characterized in that the lens (1) comprises a sensor (80) configured to sense a movement of the person wearing the lens (1), and to provide an output signal in response to a pre-determined movement of said person, wherein particularly said movement is a movement of an eyelid (4) of an eye (2) of said person.

24. The lens according claim 23, characterized in that the sensor (80) is one of: a photosensitive element, a pressure sensing element, a capacitive sensing element, a thermal sensor, particularly a resistor.

25. The lens according to claim 23, characterized in that the sensor (80) is configured to sense a deformation of the lens (1), wherein the sensor (80) is built into the actuator (70), or formed by the actuator (70), or comprises parts thereof.

26. The lens according to claim 1, characterized in that the lens (1) further comprises a processing unit (90) that is configured to actuate the at least one actuator (70) in response to the output signal provided by the sensor (80) or in response to an output signal provided by an external device (81), wherein particularly the at least one actuator (70) is actuated by applying said voltage to said electrodes (71, 72) of the at least one actuator (70).

27. The lens according to claim 13, characterized in that the lens (1) comprises an electric energy source (110), particularly a battery, wherein particularly said electric energy source (110) is configured to be charged by means of one of: inductive charging; light, wherein particularly the lens (1) comprises a solar cell (120) or a photo diode (120); using the thermoelectrical effect, wherein particularly the lens (1) comprises a Peltier element (130); using an eyelid movement or a movement of an eye, wherein particularly the lens (1) comprises a flexible capacitance (140) for transforming said eyelid movement or said movement of the eye into electrical energy; using the reverse electro-osmotic effect by pumping liquid through a membrane (430, 431).

28. The lens according to claim 12, characterized in that said surfaces (200, 100) are configured to stick to each other through a compressive force of the at least one actuator (70).

29. The lens according to claim 1, characterized in that the back side (12) of the base element (10) is configured to be placed on the surface of the eye (2) such that said back side (12) contacts said surface of the eye (2), or that the front side (21) of the membrane (20) is configured to be placed on the surface of the eye (2) such that said front side (21) contacts said surface of the eye (2).

30. The lens according to claim 1, characterized in that the reservoir volume (42) is positioned in an upper or a lower half of the lens (1), so that the reservoir volume (42) is compressible by an onset of an eyelid movement of an eye (2) of the person when the lens (1) is arranged on the pupil (3) of said eye (2), so as to pump liquid from the reservoir volume (42) into the lens volume (41) for increasing the curvature of the curvature-adjustable area (23) of the membrane (20).

31. The lens according to claim 30, characterized in that the reservoir volume (42) is formed by at least one reservoir (42a, 42b) which is connectable via at least one channel (43a, 43b) (41) to the lens volume (41), which at least one channel (42a, 42b) extends along a periphery of the lens volume (41).

32. The lens according to claim 31, characterized in that said at least one channel (43a, 43b) is connectable to the lens volume (41) via a valve (43) which is arranged in a lower half or in an upper half of the lens (1), particularly such that the valve (43) faces the at least one reservoir (42a, 42b) and/or such that the lens volume (41) is arranged between the at least one reservoir (42a, 42b) and the valve (43).

33. The lens according to claim 31, characterized in that the at least one reservoir (42a, 42b) comprises a valve (430, 431) via which the at least one reservoir is connected to the at least one channel (43a, 43b).

34. The lens according to claim 32, characterized in that the lens (1) comprises an energy source (110) that is electrically connected to the valve (43) for providing energy to the valve (43) in order to open or close the valve (43).

35. The lens according to claim 34, characterized in that the lens (1) comprises a sensor (80) for detecting a movement, particularly an eyelid movement, which sensor (80) is connected to the valve (43) or to the energy source (110), wherein the sensor (80) is configured to provide an output signal when said movement is detected by the sensor (80) and to provide the output signal to the valve (43) or to the energy source (110) for controlling the valve (43), particularly for closing or opening said valve (43).

36. The lens according to one of the claim 32, characterized in that the valve (43) is one of:
a valve (43) comprising an osmotic membrane (430, 431) forming a wall of the at least one reservoir, which osmotic membrane is configured to open and to allow the liquid (50) to pass through it depending on a voltage applied to the osmotic membrane;
a valve (43) comprising at least two electrodes for opening or closing the valve;
a valve (43) comprising a member (44) out of a shape memory alloy or a phase change material for opening or closing the valve;
a valve (43) comprising an electromagnetic actuator for opening or closing the valve;
a valve (43) comprising a magnet that is configured to be moved by a another magnet for opening or closing the valve.

37. The lens according to claim 1, characterized in that the lens (1) comprises a pump (150) which comprises the reservoir volume (42), wherein the pump (150) is configured to empty the reservoir volume (42) by moving a region (20a) of said membrane (20) covering the reservoir volume (42) into a dent (42c) forming at least a part of said reservoir volume (42), which dent is formed in the base element (10).

38. The lens according to claim 37, characterized in that the pump (150) is configured to generate a force for moving said region (20a) of the membrane (20) into the dent (42c), wherein for generating said force, said region (20a) of the membrane (20) forms a flexible and stretchable, electrically conducting electrode (20b), and the base element (10) forms at least one corresponding counter electrode (10b); or wherein for generating said force the pump (150) comprises a member (44) formed out of a shape memory alloy, which is configured to be heated, particularly by an electric current.

39. The lens according to claim 37, characterized in that a channel (42d) via which the reservoir volume (42) is connected to the lens volume (41) leads to a lowest area (42e) of a bottom (42f) of said dent (42c) of the reservoir volume (42), wherein said channel (43d) is configured to be automatically sealed when said region (20a) of the membrane (20) is moved into the dent (42c).

40. The lens according to claim 39, characterized in that when said channel (42d) is sealed, reentry of liquid (50) into the reservoir volume (42) is blocked at an intersection (42g) of the channel (42d) and the reservoir volume (42).

41. The lens according to claim 39, characterized in that the pump (150) is configured to keep the channel (42d) in its sealed state by pinning said region (20a) of the membrane (20) to an area (42e) on the bottom (42f) of said dent (42c) of the reservoir volume (42) using the electrode (20b) of the membrane (20) on one side and on the other side said counter electrode (10b) and/or a central electrode (10c) that is arranged at the center of the bottom (42f) of the dent (42c) and surrounded by said counter electrode (10b); or by using said member (44).

42. The lens according to claim 39, characterized in that the sealed channel (42d) is configured to open at a certain back pressure, which initiates liquid back flow and refilling of the reservoir volume.

43. The lens according to claim 1, characterized in that the lens (1) comprises a channel (160d) for providing a flow connection between the reservoir volume (42) and the lens volume (41), wherein the lens (1) comprises a valve (160) for opening or closing said channel (160d), wherein said channel (160d) extends through a dent (160c) of the valve (160) formed in the base element (10), which dent (160c) is covered by a region (20a) of said membrane (20), wherein the valve (160) is configured to open or block said channel (160d) by moving a region (20a) of said membrane (20) covering the dent (160c) into the dent (160c).

44. The lens according to claim 43, characterized in that the valve (160) is configured to generate a force for moving said region (20a) of the membrane (20) into the dent (160c) of the valve (160), wherein for generating said force said region (20a) of the membrane (20) forms a flexible and stretchable, electrically conducting electrode (20b), and the base element (10) forms at least one corresponding counter electrode (10b); or wherein for generating said force the valve (160) comprises a member (44) formed out of a shape memory alloy, which is configured to be heated, particularly by an electric current.

45. The lens according to claim 43, characterized in that said channel (160d) is configured to be automatically blocked when said region (20a) of the membrane (20) is moved into the dent (160c) of the valve (160).

46. The lens according to claim 45, characterized in that when said channel (160d) is blocked, reentry of liquid (50) into the dent (160c) of the valve is blocked at intersections (160g) of the channel (160d) and the dent (160c).

47. The lens according to claim 45, characterized in that the valve (160) is configured to keep the channel (160d) in its blocked state by pinning said region (20a) of the membrane (20) to an area (160e, 160g) on the bottom (160f) of said dent (160c) of the valve (160) using the electrode (20b) of the membrane (20) on one side and on the other side at least one of: said counter electrode (10b), a central electrode (10c) that is arranged at the center of the bottom (160f) of the dent (160c) and surrounded by said counter electrode (10b), a first sealing line electrode (10d) extending along an intersection (160g) between the channel (160d) and the dent (160c), a second sealing line electrode (10e) extending along a further intersection (160g) between the channel (160d) and the dent (160c), wherein said sealing line electrodes (10d, 10e) are separated from the central electrode (10c) by a gap (10f).

48. The lens according to claim 43, characterized in that the valve (160) is configured to open at a certain pressure, which allows passage of liquid (50) between the reservoir volume (42) and the lens volume (41).

49. The lens according to claim 37, characterized in that the membrane (20) or at least a region (20a) thereof is configured to be pushed down by an eyelid or finger of a user of the lens (1) in order to assist in pumping liquid (50) from the reservoir volume (42, 42a, 42b) to the lens volume (41) and/or from the lens volume into the reservoir volume.

50. The lens according to claim 1, characterized in that the reservoir volume (42) is covered by a bistable region (20a) of said membrane (20), wherein said region (20a) is movable with respect to the base element (10) from a first stable state to a second stable state and vice versa, wherein in the first state the reservoir volume (42) is larger than in the second state, and wherein when said region (20a) is moved from the first state to the second state, liquid (50) flows from the reservoir volume (42) into the lens volume (41), and wherein when the region (20a) is moved from the second state to the first state, liquid flows from the lens volume (41) back to the reservoir volume (42).

51. The lens according to claim 50, characterized in that the lens (1) comprises a channel (43) connecting the reservoir volume (42) to the lens volume (41) to allow liquid (50) to flow from the lens volume (41) to the reservoir volume (42) and vice versa.

52. The lens according to claim 50, characterized in that the reservoir volume (42) comprises a circular shape or a ring shape extending around the lens volume (41).

53. The lens according to claim 50, characterized in that said region (20a) is configured to flip from one stable state to the other stable state when sufficient pressure is applied to a concave or convex surface of said region (20a), wherein said region (20) is configured to be actuated manually in order to move it from one state to the other, particularly by means of a finger or an eyelid of a person.

54. The lens according to claim 50, characterized in that said region (20a) is given a convex or concave shape using molding or thermoforming for providing said bi-stable state.

55. The lens according to claim 50, characterized in that said region (20a) is made out of an elastomer or comprises an elastomer.

56. The lens according to claim 50, characterized in that a portion of the membrane (20) or said region (20a) is made of a metal, or a polymer, or an elastomer, or a heterogeneous structure of at least two materials.

57. System comprising a lens (1) according to claim 1 and a container (300) for storing the lens (1) when the lens (1) is not placed on the surface of the eye (2) of a person, wherein said container (300) comprises an electrically conducting coil (302) for charging an energy source (110) or battery (110) of the lens (1) by means of induction.

58. Method for manufacturing a lens (1), particularly a contact lens (1), particularly according to claim 1, comprising the steps of
Providing a base element (10),
Providing a transparent and elastically deformable membrane (20) comprising a ring member (30) connected to or integrated into a back side (22) of the membrane (20),
Optionally releasing the membrane (20) from one or several sacrificial parts, which particularly stabilize the membrane (20) for handling the membrane prior to assembly,
Bonding the base element (10) to the membrane (20) and thereby forming a lens volume (41) and a reservoir volume (42) of the lens (1),
Optionally releasing the base element (10) from sacrificial structures, particularly from a regular array of small pillars, which particularly help to avoid a contact between the base element (10) and the membrane (20) in a middle optical zone of the membrane (20) and/or in actuator regions (42) and/or in channel regions (43), prior to filling of the lens volume (41) with a transparent liquid (50), and
Filling said lens volume (41) and said reservoir volume (42) with a transparent liquid (50).

59. The method according to claim 58, wherein one of the following is applied to the membrane (20) and/or the base element (10): a coating, at least one electrode (71, 72), an insulation layer (73), an anti-stiction layer.

60. Method according to claim 58, characterized in that said filling is conducted using diffusion and osmotic pressure after said bonding has been performed.

61. Method according to claim 58, characterized in that said filling is conducted before said bonding, wherein said liquid (50) is filled into a dent (51) formed by the membrane (20), wherein thereafter said bonding is conducted, and wherein the lens volume (41) and/or reservoir volume (42) is freed from gas (53) residing therein after said bonding.

62. The method according to claim 58, characterized in that the ring member (30) is connected to the deformable membrane (20) by plasma bonding.

63. The method according to claim 58, characterized in that the ring member (30) is formed as an integral part of the membrane (20), wherein the ring member is stiffened by means of irradiating it with ultraviolet light, or wherein the membrane is softened by irradiating it with ultraviolet light.

64. The method according to claim 58, characterized in that the ring member (30) is formed as an integral part of the membrane (20), wherein a primer is applied to the mold in which the ring member is formed, which primer is designed to chemically stiffen the ring member (30) during molding of the membrane (20) and integral ring member (30).
